(19) United States
(12) Patent Application Publication
(10) Pub. No.: US 2015/0320394 A1
(43) Pub. Date: Nov. 12, 2015

(54) TORIC FOCUSING FOR RADIATION FORCE APPLICATIONS

(71) Applicant: University of Washington, Seattle, WA (US)

(72) Inventors: Bastien Arnal, Paris (FR); Thu-Mai Nguyen, Villejuif (FR); Matthew O'Donnell, Seattle, WA (US)

(21) Appl. No.: 14/710,464

(22) Filed: May 12, 2015

Related U.S. Application Data
(60) Provisional application No. 61/991,740, filed on May 12, 2014.

(51) Int. Cl. A61B 8/08; A61B 8/00; A61B 5/00

(52) U.S. Cl. CPC A61B 8/485; A61B 5/0066; A61B 8/4488

(57) ABSTRACT

The present disclosure provides systems, methods, and devices for improved generation and application of radiation force to a material. In one aspect, a system for producing an acoustic radiation force to generate displacement in a material comprises an ultrasonic transducer array, one or more processors, and memory. The memory can comprise instructions that, when executed by the one or more processors, cause the system to generate a push acoustic energy focused to a push focal region in the material using the ultrasonic transducer array, so as to produce the acoustic radiation force to generate displacement in the material. The push focal region can comprise a first width along a first direction transverse to a direction of propagation of the push acoustic energy greater than a second width along a second direction transverse to the direction of propagation of the push acoustic energy.

### CROSS-REFERENCE

This application claims the benefit of U.S. Provisional Application No. 61/991,740, filed May 12, 2014, which application is incorporated herein by reference in its entirety.

### STATEMENT AS TO FEDERALLY SPONSORED RESEARCH

This invention was made with government support under Grant number 1 R01 EB 016034-01 awarded by the National Institutes of Health. The government has certain rights in the invention.

### BACKGROUND

Several prior elastography methods have been applied to clinical problems. For many applications, dynamic elastography has produced higher resolution elasticity images that are more robust than static methods. A few different approaches can be used to induce and measure a transient deformation within tissue. Early studies used an external vibrator to produce deformations and ultrasound or magnetic-resonance imaging to detect induced displacements. Ultrasound methods and apparatus such as Acoustic Radiation Forced Imaging (ARFI) and Shear Wave Imaging (SWI) can be used to transiently deform tissue remote from the ultrasound source and to measure the transient deformation.

The prior methods and apparatus for imaging with ARFI and SWI have been less than ideal in at least some instances. For example, prior methods for generating acoustic radiation force may utilize focusing geometries that are less than ideal for efficient force delivery. As the amount of energy used for transient tissue deformation can be limited for safety, the prior methods and apparatus for focusing spots can result in less than ideal amounts of transient tissue deformation. Additionally, although prior methods and apparatus may utilize an ultrasound transducer for generating acoustic radiation force and imaging of the resultant tissue response, the prior methods and apparatus have resulted in less than ideal force delivery and displacement measurement. Although prior ARFI has measured tissue response within a focal region, prior ARFI may have resulted in less than ideal focusing of the spot in the focal region and less than ideal subsequent imaging around the focal region in at least some instances. Although prior SWI has captured the propagation of a shear wave to retrieve tissue elasticity, the focusing of the shear wave generating ultrasound spot and subsequent imaging can be less than ideal, and the prior SWI instrumentation more complex than would be ideal. Although Supersonic Shear Imaging (SSI) is an implementation of SWI that has been adopted clinically for breast cancer imaging and other applications, typical focus patterns used for both acoustic force generation and imaging with prior SSI methods and apparatus can be less than ideal.

Although prior optical coherence tomography (OCT)-based methods and apparatus have been used to image displacements and produce elastography measurements, the prior OCT based methods and apparatus have been less than ideal in at least some respects. For example, prior OCT based methods and apparatus may have produced less than ideal acoustic radiation force focusing with less than ideal imaging in at least some instances.

Improved methods and apparatus are needed for measuring transient tissue deformation. Ideally, such improved methods and apparatus will provide improved measurements with safe amounts of energy, improved beam focusing, improved imaging and decreased complexity.

### SUMMARY

The present disclosure provides systems, methods, and devices for improved generation and application of radiation force to a material. The systems, methods and devices disclosed herein provide improved measurement of transient tissue deformation with safe amounts of energy and decreased complexity. In some embodiments, a system comprises an ultrasonic transducer array used to produce acoustic radiation force for generating displacement of a material. The array can be programmed to focus the acoustic energy to a focal region having a first width along a first direction transverse to the direction of propagation of the acoustic energy (e.g., an elevational width) that is greater than a second width along a second direction transverse to the direction of propagation of the acoustic energy (e.g., an azimuthal width) in order to produce the acoustic radiation force with improved efficiency, increased transient tissue deformation and improved measurement and decreased complexity. For instance, the transducer array can be a programmable phased array and the system can apply a set of delays to the phased array to produce toric focusing of the acoustic energy. Toric focusing can elicit larger material displacements with reduced acoustic pressure compared to other focusing geometries, thus improving the efficiency of acoustic radiation force delivery. In some embodiments, an ultrasonic transducer array is dynamically reconfigurable to provide different focal geometries shaped for acoustic radiation force generation and acoustic imaging, respectively, thereby providing both efficient force delivery and sensitive displacement and detection with a single array. The embodiments disclosed herein can be combined in many ways and can be used in conjunction with other types of imaging modalities such as optical coherence tomography (OCT), thus providing greater flexibility and applicability.

In one aspect, a system for producing an acoustic radiation force to generate displacement in a material is provided. The system can comprise an ultrasonic transducer array comprising a plurality of transducer elements arranged along a first direction and a second direction, one or more processors, and memory. The memory can comprise instructions that, when executed by the one or more processors, cause the system to generate a push acoustic energy using the ultrasonic transducer array. The push acoustic energy can be focused to a push focal region in the material so as to produce the acoustic radiation force to generate displacement in the material. The push focal region can comprise a first width along a first direction transverse to a direction of propagation of the push acoustic energy greater than a second width along a second direction transverse to the direction of propagation of the push acoustic energy.

The geometry of the push focal region can be varied as desired. In some embodiments, the push focal region comprises one or more of an asymmetrical geometry, an aspherical geometry, or a planar geometry. In some embodiments, the first width comprises an elevational width and the second width comprises an azimuthal width. The elevational width can be at least 8 times greater than the azimuthal width.

In some embodiments, the push acoustic energy produces a peak acoustic radiation pressure in the material of less than or equal to about 4 MPa. The push acoustic energy can comprise a frequency of at least 20 MHz. The acoustic radiation force produced by the push acoustic energy can produce shear waves that generate the displacement of the material.

Various types of ultrasonic transducer arrays are suitable for use with the embodiments herein. In some embodiments, the ultrasonic transducer array comprises one or more of a ring array, an annular array, a 1.25 D array, a 1.5 D array, a 1.75 D array, or a 2 D array of transducer elements. The plurality of transducer elements can each comprise a characteristic dimension less than an ultrasonic wavelength at a primary operating frequency of the ultrasonic transducer array. In some embodiments, the ultrasonic transducer array comprises a programmable phased array and the push acoustic energy is generated by applying a set of delays and amplitudes to the programmable phased array configured to produce toric focusing of the push acoustic energy.

Optionally, the instructions can further cause the system to generate an imaging acoustic energy using the ultrasonic transducer array. The imaging acoustic energy can be focused to an imaging focal region in the material so as to measure the displacement of the material generated by the acoustic radiation force. The imaging focal region comprises a different geometry than the push focal region. For instance, the push focal region can comprise a geometry configured to produce the acoustic radiation force and the imaging focal region can comprise a geometry configured to measure the displacement of the material. In some embodiments, the imaging focal region is generated by spherical focusing of the ultrasonic transducer array during a transmit phase and dynamic focusing of the ultrasonic transducer array during a receive phase to produce a real-time image.

Some embodiments of the present disclosure enable generation of push acoustic energy and imaging acoustic energy with a single ultrasonic transducer array. The generation of the push acoustic energy can be temporally coordinated with the generation of the imaging acoustic energy. For example, the imaging acoustic energy can be generated no more than about 1 μs after generating the push acoustic energy. In some embodiments, the ultrasonic transducer array comprises a programmable phased array, and the push acoustic energy is generated by applying a first set of delays and amplitudes to the programmable phased array in order to focus the push acoustic energy. The imaging acoustic energy can be generated by applying a second, different set of delays and amplitudes to the programmable phased array in order to focus the imaging acoustic energy. The first set of delays and amplitudes can be arranged to produce toric focusing of the push acoustic energy and the second set of delays and amplitudes can be arranged to produce spherical focusing of the imaging acoustic energy.

In some embodiments, the system further comprises an imaging device. The instructions can further cause the system to measure the displacement of the material generated by the acoustic radiation force using the imaging device. The imaging device can be separate from the ultrasonic transducer array. For instance, the imaging device can comprise an optical coherence tomography (OCT) imaging device. Optionally, the imaging device can be aligned with the ultrasonic transducer array in order to measure the displacement of the material. The generation of the push acoustic energy using the ultrasonic transducer array can be temporally coordinated with the measurement of the displacement of the material using the imaging device. The instructions can further cause the system to move the push focal region to a plurality of different positions in the material.

In another aspect, a method comprises providing a system as in any of the embodiments presented herein.

In another aspect, a method for producing an acoustic radiation force to generate displacement in a material is provided. The method can comprise generating a push acoustic energy focused to a push focal region in the material using an ultrasonic transducer array. The push acoustic energy can produce the acoustic radiation force to generate displacement in the material. The push focal region can comprise a first focal width along a first direction transverse to a direction of propagation of the acoustic energy greater than a second focal width along a second direction transverse to the direction of propagation of the acoustic energy.

In another aspect, one or more non-transitory computer-readable storage media are provided. The one or more non-transitory computer-readable storage media can have stored thereon executable instructions that, when executed by one or more processors of a system for producing an acoustic radiation force to generate displacement in a material, cause the system to generate a push acoustic energy using an ultrasonic transducer array. The push acoustic energy can be focused to a push focal region in the material so as to produce the acoustic radiation force to generate displacement in the material. The push focal region can comprise a first focal width along a first direction transverse to a direction of propagation of the acoustic energy greater than a second focal width along a second direction transverse to the direction of propagation of the acoustic energy.

Other objects and features of the present disclosure will become apparent by a review of the specification, claims, and appended figures.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an ultrasonic transducer producing an acoustic radiation force, in accordance with embodiments;

FIG. 2 illustrates a focal region generated by spherical focusing of an ultrasonic transducer, in accordance with embodiments;

FIG. 3 illustrates a focal region generated by toric focusing of an ultrasonic transducer, in accordance with embodiments;

FIG. 4 illustrates a system for producing and measuring displacement of a material using an ultrasonic transducer array, in accordance with embodiments;

FIG. 5 illustrates a timing diagram for controlling transmission of acoustic energy for producing and measuring displacement of a material, in accordance with embodiments;

FIG. 6 illustrates a method for producing an acoustic radiation force to elicit displacement and/or deformation of a material, in accordance with embodiments;

FIG. 7 illustrates a system for producing and measuring displacement of a material using an ultrasonic transducer array and a separate imaging device, in accordance with embodiments;

FIG. 8 illustrates producing an acoustic radiation force to elicit displacement and/or deformation of a material, in accordance with embodiments;

FIGS. 9A and 9B illustrates toric lens geometry for a focal distance ratio of 1.2, in accordance with embodiments;

FIG. 10A illustrates configuration and alignment of a single transducer element and imaging array for quantification of shear wave amplitudes, in accordance with embodiments;

FIG. 10B illustrates configuration and alignment of a single transducer element and imaging array for shear wave elastography experiments, in accordance with embodiments;

FIGS. 11A through 11C illustrate simulated and experimental acoustic fields, in accordance with embodiments;

FIGS. 12A and 12B illustrate displacements obtained using spherical and toric ultrasound focal spots, in accordance with embodiments;

FIG. 13 illustrates normalized displacement amplitude as a function of elevational focal width, in accordance with embodiments;

FIGS. 14A and 14B illustrate snapshots of shear wave propagation induced using a spherical and a toric ultrasound focal spot, in accordance with embodiments;

FIGS. 15A through 15D illustrate shear wave speed maps obtained using a spherical and a toric ultrasound focal spot at two different peak pressures, in accordance with embodiments; and

FIG. 15E illustrates a B-mode image of the phantom in FIGS. 15A through 15D, in accordance with embodiments.

### DETAILED DESCRIPTION

Systems, methods, and devices are provided herein for performing ultrasound elastography in order to measure mechanical properties (e.g., elasticity) of a material, such as a tissue (e.g., a tissue within a living organism such as a human being). In some embodiments, an elastography procedure involves using an ultrasonic transducer to transmit acoustic energy into a material. The acoustic energy creates acoustic pressure in the material, thus producing acoustic radiation force. The application of acoustic radiation force to a material can elicit a mechanical response, such as displacement and/or deformation of the material. The material displacement and/or deformation can propagate through the material along a transverse direction as shear waves, and the mechanical properties of the material can be measured by observing the propagation of the shear waves. Acoustic radiation force that elicits material displacement and/or deformation may be referred to herein as a “push,” and the acoustic energy used to produce such a push may be referred to herein as “push acoustic energy.” Push acoustic energy can be configured to displace and/or deform the material without damaging and/or destroying the material.

The embodiments disclosed herein are well suited for many types of elasticity measurements and can be used with one or more of many materials. The embodiments disclosed herein are well suited for use with ophthalmic applications such as measurement of the stiffness of the lens of the eye to determine the presence of presbyopia, and the stiffness of tissues of the eye that may be related to glaucoma, for example.

The embodiments disclosed herein can be combined in many ways, and U.S. Provisional Application No. 61/991,740, filed May 12, 2014, which has been previously incorporated herein by reference in its entirety, describes methods and apparatus suitable for incorporation in accordance with some embodiments of the present disclosure.

As used herein like characters refer to like elements.

As used herein “and/or” encompasses either or both of two or more stated possibilities. For example, A and/or B encompasses A alone, B alone, and A and B together.

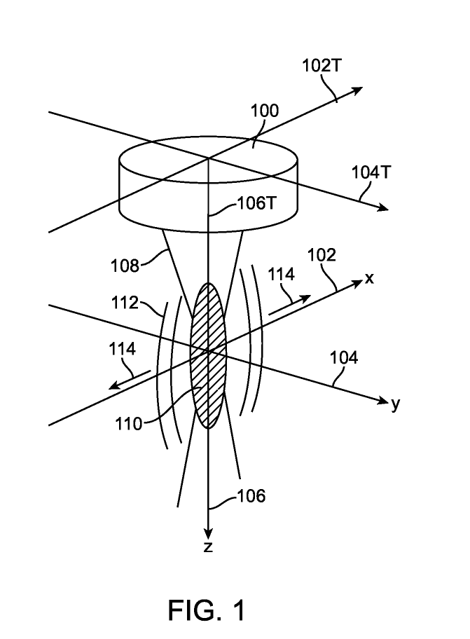

FIG. 1 illustrates an ultrasonic transducer 100 producing a transient tissue deformation with an acoustic radiation force, in accordance with embodiments. The orientation of the transducer 100 can be used to define a coordinate system with three orthogonal axes: an x-axis 102 (azimuth direction), a y-axis 104 (elevation direction), and a z-axis 106 (axial direction). The axial (z-direction) is aligned with the direction of propagation of acoustic energy 108 from the transducer 100, while the azimuth (x-direction) and elevation (y-direction) are orthogonal to the direction of propagation of the acoustic energy 108. The geometry of the transducer 100 can be defined with respect to three orthogonal transducer axes: an x-axis 102T, a y-axis 104T, and a z-axis 106T. For example, in embodiments where the transducer 100 is an array transducer as discussed herein, the axes 102T and 104T can define first and second directions for the elements of the array. The transducer axes 102T, 104T, 106T can be aligned with the corresponding coordinate axes 102, 104, 106. For example, the transducer axis 106T can be coaxial with the axis 106, and the transducer axes 102T and 104T can be parallel to the axes 102 and 104, respectively.

The transducer 100 can focus the acoustic energy 108 to a focal region 110 at a position along the axial direction. Material within the focal region 110 may be exposed to larger amounts of acoustic pressure than material outside the focal region 110. Accordingly, the focal region 110 can be a “push focal region” acting as a “push source” of acoustic radiation force that produces shear waves 112 in the material originating in and propagating outward from the focal region 110. The transducer 100 can be used to image the material displacements resulting from the propagating shear waves 112. In some embodiments, the azimuthal (x-z) plane serves as the imaging plane, such that the propagation of the shear waves 112 is observed along the azimuthal direction, as indicated by arrows 114.

Various types of ultrasonic transducers are suitable for use with the embodiments presented herein. For example, the ultrasonic transducer can be a single element transducer (e.g., linear, planar, spherical, etc.). Alternatively, the ultrasonic transducer can be an ultrasonic transducer array having a plurality of ultrasonic transducer elements. For example, an ultrasonic transducer array can include at least 64 transducer elements, or within a range from 64 to 4096 transducer elements. The spatial arrangement of the transducer elements of an ultrasonic transducer array can be varied as desired. In some embodiments, the transducer elements are arranged along a single direction (e.g., in a single row or column), thus forming a linear or 1 D array. Alternatively, the transducer elements can be arranged along more than one direction, e.g., along a first direction and a second direction, such that the dimensionality of the array is greater than 1. For example, the transducer elements can be arranged in multiple rows and columns so as to form 1.25 D, 1.5 D, 1.75 D, or 2 D arrays, depending on the number of elements used in the second direction. As another example, the transducer elements can be arranged according to other geometries with dimensionalities greater than 1, such as a ring array or an annular array.

The characteristics of the ultrasonic transducer array can be varied as desired. For example, the ultrasonic transducer array can be characterized by the frequency of the acoustic energy it produces, also known as the “operating frequency.” The operating frequency of the ultrasonic transducer array can vary based on the particular application. In some embodiments, the ultrasonic transducer array is configured to operate at frequencies suitable for elastography applications and/or imaging applications. For instance, the ultrasonic transducer array can have a primary operating frequency of about 8 MHz, or within a range from about 1 MHz to about 30 MHz. In some embodiments, the ultrasonic transducer array 104 has a primary operating frequency of at least 1 MHz, at least 5 MHz, at least 10 MHz, or at least 20 MHz.

The dimensions of the ultrasonic transducer array can be varied as desired. The ultrasonic transducer array and/or the elements thereof can have a characteristic dimension. “Characteristic dimension” may be used herein to refer to the element size (e.g., length and/or width) of an individual transducer element in the array. Alternatively or in combination, “characteristic dimension” may be used herein to refer to the total aperture width of the transducer array. In some embodiments, the characteristic dimension (e.g., element size) is related to the primary operating frequency of the array. For example, the characteristic dimension (e.g., element size) of some or all of the elements in the transducer array can be less than the ultrasonic wavelength at the primary operating frequency of the array, such as about half of the wavelength at the primary operating frequency or less. In some embodiments, the ultrasonic transducer array has a length and/or width that is less than or equal to 32 wavelengths at the primary operating frequency.

In some embodiments, the ultrasonic transducer array is a programmable phased array where each transducer element can be separately pulsed according to a desired timing. The shape and/or steering of the generated acoustic energy can thus be controlled by electronically controlling the delay time applied to each transducer element. Similarly, controlled delays can be used to focus the acoustic energy generated by the array according to a desired focusing geometry, as discussed further herein. The set of delays that would produce acoustic energy having a particular shape, steering, and/or focusing geometry can be calculated using methods known to one of ordinary skill in the art. Although reference is made to temporal delays, other adjustments to the waveform can be provided, such as temporal advancements that can be provided to the waveform used to drive the phase array. Alternatively or in combination, adjustments in the amplitude of the signal at each transducer element (also referred to herein as “amplitude weight”) can be used to produce a desired focusing geometry.

The ultrasonic transducers presented herein may be characterized according to their focusing geometries. The term “focusing geometry” (also referred to herein as “focus geometry” or “focal geometry”) may be used herein to refer to the manner in which the ultrasonic transducer focuses the generated acoustic energy. For example, “spherical focusing geometry” can refer to the focusing of acoustic energy that is produced by a spherical transducer or spherical acoustic lens, or an equivalent thereof (e.g., a phased array with programmed delays and/or amplitudes that mimic the effect of a spherical lens). The acoustic energy generated by the ultrasonic transducer can be focused to a focal region with a geometry (e.g., size, shape) determined by the particular focusing geometry of the transducer. Various types of focal region geometries are suitable for use with the embodiments herein, including spherical geometries, ellipsoidal geometries, cylindrical geometries, planar geometries, symmetrical geometries, aspherical geometries, and/or aspherical geometries. Certain focal region geometries may be optimal and/or preferred for certain applications. For instance, spherical focusing or cylindrical focusing may be optimal and/or preferred for transmitting acoustic energy for imaging applications, as discussed further herein.

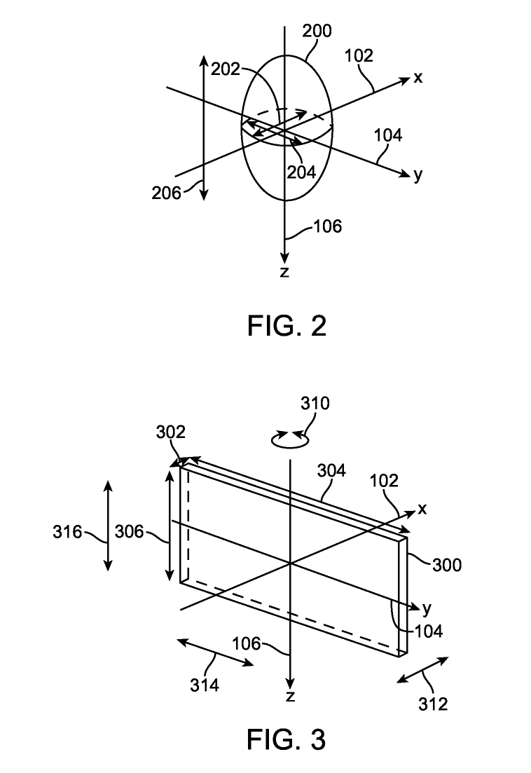

FIG. 2 illustrates a focal region 200 generated by spherical focusing of an ultrasonic transducer, in accordance with embodiments. The focal region 200 can be defined with respect to x (azimuthal), y (elevational), and z (axial) axes, which may correspond to the axes 102, 104, and 106 previously discussed herein with respect to FIG. 1. The focal region 200 has an ellipsoidal geometry, with the longitudinal axis of the ellipsoid aligned with the axial direction (z). In some embodiments, the azimuthal width 202 of the focal region 200 is approximately equal to the elevational width 204, such that the shape profile of the focal region 200 in the azimuthal-elevational (x-y) plane is circular. The axial length 206 of the focal region 200 can be greater than the azimuthal width 202 and the elevational width 204, such that the shape profile of the focal region 200 in the azimuthal-axial (x-z) plane and elevational-axial (y-z) plane is elongated along the axial direction.

Various methods and apparatus can be used to control the focusing geometry of an ultrasonic transducer. For example, the focusing geometry of an ultrasonic transducer can be physically controlled, e.g., by altering the shape of the transducer itself and/or using an acoustic lens coupled to the transducer. Alternatively or in combination, the focusing geometry can be electronically controlled, e.g., by controlling the set of delays and/or amplitudes applied to the elements of a programmable phased array. Electronic control may advantageously enable dynamic reconfiguration to different focusing geometries, as discussed further herein.

In some embodiments, controlling the focusing geometry of an ultrasonic transducer involves controlling the focusing of the acoustic energy with respect to the azimuthal direction and/or the elevational direction. Different transducer types may afford differing degrees of control of azimuthal and/or elevational focusing (e.g., the width of the focal region (“focal width”) along the azimuthal and/or elevational directions). For example, certain methods may use one of two main types of ultrasonic transducers in order to reach the required acoustic power to induce radiation force: spherically-focused, high-intensity transducers or imaging arrays. High-intensity sources can be single elements or phased array systems combining multiple transducers disposed on a spherical shell. A spherically-focused transducer may be symmetrical such that the focusing in the elevational direction is the same or similar to the focusing in the azimuthal direction (e.g., the elevational focal width is the same or similar to the azimuthal focal width). A symmetrical focusing geometry such as a spherical focusing geometry may not allow for independent modification of the focus along the elevational and azimuthal directions (e.g., the elevational focal width cannot be modified independently from the azimuthal focal width). Imaging arrays (linear, curved, cardiac, etc.) may be primarily designed for clinical applications by finding the best tradeoff between resolution, side lobe reduction, number of channels, and electrical impedance of the elements. In some embodiments, a standard imaging probe includes a 128-element, 1-D array with an elevation height of 5 to 20 mm. Focusing in the elevation direction can be done using an acoustic lens with high f-numbers (typically 3 to 6) compared to 1 to 2 used in azimuth. Thus, focusing may be weaker in the elevation direction than in the plane of the elements.

The focusing geometry of an ultrasonic transducer can influence the efficiency with which it produces material displacement and/or deformation for elastography applications. Efficiency may be used herein to denote the magnitude of the material response (e.g., amount of displacement and/or deformation) achieved per amount of acoustic radiation pressure generated in the material by the transducer. Increased efficiency may be beneficial for improving the precision and accuracy of elastography measurements with reduced acoustic radiation pressure. As with any measurement technique, good signal-to-noise ratio (SNR) can influence the precision and accuracy of the measurement results. Radiation force can be proportional to the acoustic intensity. However, in the context of medical elastography, FDA regulations may limit the acoustic pressure via the mechanical index (MI), defined as

$$M I = \frac{P}{\sqrt{f}}$$

where P is the peak acoustic pressure and f is the frequency in MHz. For example, the MI may be 1.9 for general applications and 0.23 for ophthalmic applications. The tissue response can be based on a combination of multiple factors, including tissue biomechanical properties, the duration of the radiation force emission (also limited by FDA regulations), and/or the geometry of the radiation force source. Thus, improved efficiency may be particularly beneficial for medical elastography applications in order to improve the SNR of elastography measurements without violating MI limits or other regulatory constraints.

In some embodiments, improved efficiency is achieved by controlling the focusing geometry of the ultrasonic transducer. In some embodiments, the efficiency is improved without increasing the applied acoustic pressure, considering that the MI is a limiting factor. For example, in some embodiments, increased efficiency is achieved by controlling the focusing geometry only. Given that only the focal geometry is changed, other intensity measures such as the spatial peak temporal average intensity (ISPTA) are conserved if the MI is conserved. For any focal geometry in general, thermal limitations can be met by reducing the pulse repetition frequency or duty cycle of the radiation force excitations.

In some embodiments, increased acoustic radiation force efficiency is obtained by selectively modifying the focusing of the ultrasonic transducer along a first direction, without altering the focusing along a second (e.g., orthogonal) direction. The focusing can be modified along the first direction in order to increase the focal width along that direction only, without modifying the focal width along the second direction. Accordingly, the focal region can have a first focal width along the first direction greater than a second focal width along the second direction. The first focal width can be at least 2 times, at least 3 times, at least 4 times, at least 5 times, at least 6 times, at least 7 times, at least 8 times, at least 9 times, at least 10 times, or at least 20 times greater than the second focal width. In such embodiments, the resultant focal region can have an asymmetrical, aspherical, and/or planar geometry.

In some embodiments, the first and second directions are transverse and/or orthogonal to the direction of propagation of the acoustic energy from the transducer. For example, the first direction can be an elevational direction and the second direction can be an azimuthal direction, or vice-versa. Accordingly, the present disclosure enables the focusing along the elevational direction to be modified independently from the focusing along the azimuthal direction, or vice-versa. The first and second directions can be transverse and/or orthogonal to each other. The resultant focal region can have an elevational width greater than an azimuthal width, or vice-versa.

Various methods can be used to produce a focal region having different focal widths along different directions. For instance, an ultrasonic transducer with an asymmetrical and/or aspherical focusing geometry can be used to enable independent modification of the focus along the first and second directions (e.g., elevational and azimuthal directions). In some embodiments, an ultrasonic transducer with a toric focusing geometry is used to produce a focal region with a first focal width along a first direction greater than a second focal width along a second direction. As used herein, “toric focusing geometry” can refer to the focusing of acoustic energy that is produced by a toric transducer or toric acoustic lens, or an equivalent thereof (e.g., a phased array with programmed delays and/or amplitudes that mimic the effect of a toric lens). A torically-focused tranducer can be characterized by having a focal distance along a first orientation (e.g., within an elevational-axial plane) that is different than a focal distance along a second, orthogonal orientation (e.g., within an azimuthal-axial plane).

FIG. 3 illustrates a focal region 300 produced by toric focusing of an ultrasonic transducer, in accordance with embodiments. The focal region 300 can be defined with respect to x (azimuthal), y (elevational), and z (axial) axes, which may correspond to the axes 102, 104, and 106 previously discussed herein with respect to FIG. 1. The toric focal region 300 can have a planar or substantially planar geometry. In some embodiments, the elevational width 304 of the focal region 300 is greater than the azimuthal width 302, such that the shape profile of the focal region in the azimuthal-elevational (x-y) plane is elongated along the elevational direction. For example, the elevational width 304 can be at least 2 times, at least 3 times, at least 4 times, at least 5 times, at least 6 times, at least 7 times, at least 8 times, at least 9 times, at least 10 times, or at least 20 times greater than the azimuthal width 302. The axial length 306 of the focal region 300 can be greater than the azimuthal width 302, such that the shape profile of the focal region 300 in the azimuthal-axial (x-z) plane is elongated along the axial direction. The axial length 306 may be greater than, approximately equal to, or less than the elevational width 304, as desired. The shape profile of the focal region 300 in the elevational-axial (y-z) plane can be substantially planar.

In some embodiments, the focal region 300 is produced by a phased transducer array, as discussed above and herein. The phased array can be configured to provide a toric focus as described herein. The delays of the transducer elements can be arranged in relation to a first elevation axis 102T along the transducer array and a second azimuthal axis 104T along the transducer array, in order to provide toric focusing in relation to the first axis and the second axis. The temporal delays can comprise a greater amount of time in a direction along the second axis (e.g., 104T) than a direction along the first axis (e.g., 102T) of the array, in order to provide a shorter focal distance along the first axis, for example. The resulting arrangement of delays on the transducer array can provide a substantially planar focal region 300 having a decreased focal width 302 as compared to a focal width 304, for example.

The focal region 300 can be scanned in many ways, and is not limited to any particular orientation or location. For example, focal region 300 can be scanned with one or more of a translational movement 312 along the direction of the x-axis 102, a second translational movement 314 along the direction of the y-axis 104, a third translational movement 316 along the z-axis 106, or a rotation 310 about an axis such as the z-axis 106. Alternatively or in combination, the focal region 300 can be scanned with at least partial rotation around additional axes such as x-axis 102 and/or y-axis 104. The scanning can be provided with appropriate adjustments to the timing and/or amplitude of the ultrasound waveform emitted from the ultrasound transducer array in order to provide appropriate phase of the emitted waveform to induce toric or other asymmetric focusing of the beam.

The scanning of the focal region 300 allows many types of tissue analysis in order to determine material properties of the tissue such as anisotropic properties of the tissue. Tissue response of layers of tissue such as collagenous tissue may vary with the orientation of focal region 300 in relation to the layers of tissue, and rotation of the focal region 300 can be used to interrogate anisotropic behavior of tissue based on changes to the transient deformation of tissue in response to the orientation of the focal region 300. The dimensions of the length and widths of the focal region 300 can be substantially maintained when the focal region 300 is scanned over a three dimensional volume and optionally rotated about one or more axes such as the z-axis 106. While the focal region 300 can be scanned in any pattern, in some embodiments the focal region is scanned in a predetermined scan pattern in relation to transducer array elevational axis 102T and transducer array azimuthal axis 104T, for example with one or more of a translation or a rotation as described herein. In some embodiments, the transducer array comprises external indicia such as a mark that allows the user to orient the transducer array in relation to the subject being scanned and to scan the subject with predetermined scan pattern at a desired orientation relative to the subject.

The toric focusing methods and apparatus as described herein can enable generation of acoustic radiation force with increased efficiency compared to other focusing geometries (e.g., spherical focusing). In some embodiments, toric focusing can produce shear waves in a material with peak displacements that are at least 2 times, at least 3 times, at least 4 times, at least 5 times, at least 6 times, at least 7 times, at least 8 times, at least 9 times, or at least 10 times greater than corresponding peak displacements obtained with spherical focusing at an equivalent MI. Accordingly, torically-focused transducers can be used to perform elastography with lower peak acoustic radiation pressures than transducers with other types of focusing geometries. For instance, the acoustic energy generated by a torically-focused transducer for elastography applications can produce a peak acoustic radiation pressure in the material less than or equal to about 1 MPa, 2 MPa, 3 MPa, 4 MPa, 5 MPa, 6 MPa, 7 MPa, 8 MPa, 9 MPa, or 10 MPa. Additional discussion of exemplary results that can be obtained using toric focusing is provided in Example 1 below.

The increased efficiency associated with toric focusing can improve the accuracy and precision of elastography measurements, as well as improve the efficiency of elastography-based procedures. For example, radiation force used with high-power, 2 D phased arrays may usually have a spherical geometry. Applications such as blood brain barrier opening, neurostimulation, hyperthermia or cavitation-based treatments may use magnetic resonance acoustic radiation force imaging (MR-ARFI) to monitor the focus quality and position through the skull. In some embodiments, averaging over multiple acquisitions is usually performed since the initial SNR is poor. Toric focusing could provide a higher SNR and, thus, reduce the averaging time and speed up the procedure.

The toric focusing approaches presented herein can be implemented in a variety of ways. For example, a single-element transducer with a toric surface can be used. As another example, a toric acoustic lens can be coupled to the surface of a transducer (e.g., a single-element spherical transducer) in order to achieve toric focusing without modifying the transducer itself. Such lenses can be fabricated in accordance with methods known to one of ordinary skill in the art, e.g., molded from polydimethylsiloxane (PDMS) or other suitable materials.

In some embodiments, toric focusing is implemented using an ultrasonic transducer array, such as an array with elements arranged along first and second directions (e.g., a ring array, annular array, 1.25 D array, 1.5 D array, 1.75 D array, 2 D array, etc.). The ultrasonic transducer array can be a programmable phased array, as described above and herein, and a set of delays and/or amplitudes can be applied to the programmable phased array to produce toric focusing of the generated acoustic energy. Methods for calculating the set of delays and/or amplitudes to be applied to a programmable phased array in order to achieve toric focus are known to those of ordinary skill in the art. For example, the set of delays for generating a desired toric focusing geometry using a 2 D programmable phased array can be determined as follows:

Let (i, j) be the index of the elements of the transducer array. A toric focusing can define two different focal distances, fₓ and f_{y}, using a toric surface defined with two radii (R and r). The outer surface curvature is respectively R+r and r in the two directions defining the two focal distances:

$$\{ \begin{matrix}R + r = {f}_{x} \\ r = {f}_{y}\end{matrix}$$

Choosing fₓ and f_{y}, imposes values for R and r. If the frame is taken centered on the focus lateral and elevation directions, the toric surface can be written using two angles θ and φ:

$$\{ \begin{matrix}x = ( R + r . cos ϕ ) . sin ( θ ) \\ y = r . sin ϕ \\ z = ( R + r . cos ϕ ) . cos ( θ )\end{matrix}$$

For each j index, the corresponding φ can be found so that so that:

y(j)=r·sin(φ)

φ(j)=arcsin(y(j)/r))

Then, for each i and j, the corresponding θ can be found so that:

$$θ ( i , j ) = arcsin ( \frac{x ( i )}{R + r . cos ( ϕ ( j ) )} )$$

The delay to apply on the element can correspond to the distance between the torus surface and the actual element:

$$δ t ( i , j ) = \frac{z ( i , j )}{c} = \frac{1}{c} . ( ( R . + r . cos ( ϕ ( j ) ) ) . cos ( θ ( i , j ) ) )$$

The above calculation is performed in a frame centered on the focus, but can also be performed in the probe frame using a simple 2 D translation.

In some embodiments, a phased array implementation enables a single ultrasonic transducer array to be used both for producing acoustic radiation force and for performing imaging. For example, the array can first generate a push acoustic energy for generating material displacement and/or deformation, followed by generation of imaging acoustic energy for measuring the displacement and/or deformation. Imaging acoustic energy can be configured to not produce any displacement and/or deformation when the imaging acoustic energy is applied to the material. The optimal focusing geometry for producing acoustic radiation force can be different from the optimal focusing geometry for imaging. For example, toric focusing may be optimal and/or preferred for producing acoustic radiation force, while spherical focusing may be optimal and/or preferred for imaging. The use of programmable phased transducer arrays enables the focusing geometry to be dynamically modified to produce the desired focusing geometry for each function by simply applying a different set of electronic delays and/or amplitudes. Advantageously, this approach permits the ultrasonic transducer array to be rapidly switched between functional modes (e.g., pushing and imaging) in order to achieve accurate and efficient elastography measurements.

For example, the toric focusing approaches described herein may be highly beneficial with 2 D imaging arrays. Indeed, certain embodiments enable scanners with more than one thousand channels, handling 2 D imaging arrays for 3 D real-time imaging (e.g., 4 D ultrasound). For such embodiments, toric focusing can be achieved using electronic delays and/or amplitudes, thus providing great flexibility in the focus geometry. 3 D radiation force measurements can then be optimized using a toric focusing approach by keeping a thin focus in a plane of interest to maximize the bandwidth of the shear wave and spreading the focus in the out-of-plane direction to maximize tissue response. The electronic “lens” can then be reconfigured instantaneously for the optimal geometry to detect the propagating shear wave. Overall, a programmable 2 D array (or other transducer array type) can provide improved flexibility in optimizing the focus for efficient radiation force delivery with simultaneous sensitive detection of induced displacements.

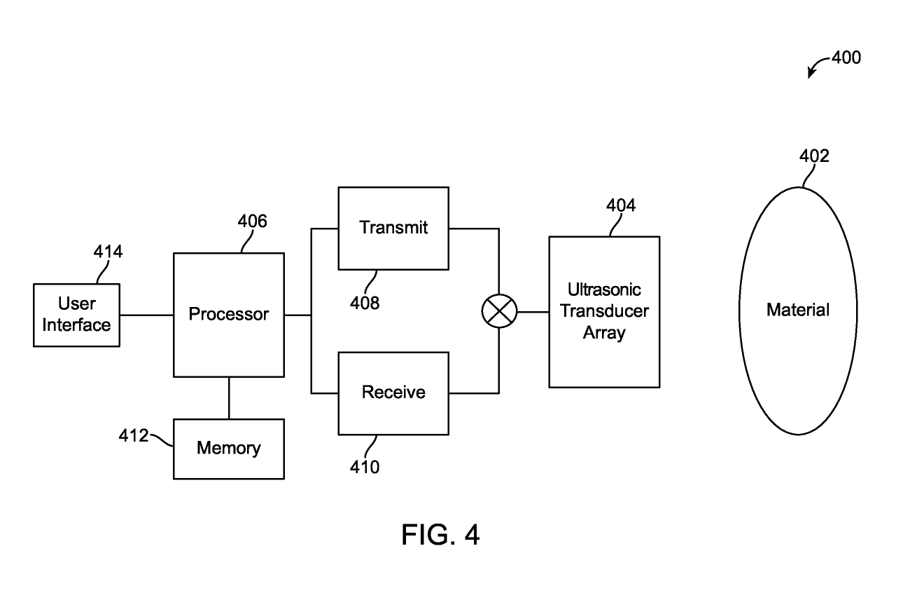

FIG. 4 illustrates a system 400 for producing and measuring displacement of a material 402 using an ultrasonic transducer array 404, in accordance with embodiments. The material 402 may comprise one or more of many materials, such as tissue, for example tissue of one or more of a lumen, vasculature, an organ, or an eye. The embodiments disclosed herein are well suited for combination with prior methods and apparatus, such as prior OCT imaging apparatus, and can be used to measure transient deformation of one or more tissues of any eye, such as one or more of a cornea, a lens, a lens capsule, a lens cortex, a lens nucleus, or drainage tissue of any eye such as a trabecular meshwork or Schlemm's canal, for example. The system 400 includes an ultrasonic transducer array 404 configured to generate and transmit acoustic energy to the material 402 during a transmit phase, and receive acoustic energy reflected from the material 402 during a receive phase. In some embodiments, the ultrasonic transducer array 404 includes transducer elements arranged along at least two directions (e.g., is a ring array, annular array, 1.25 D array, 1.5 D array, 1.75 D array, 2 D array, etc.). The system 400 also includes at least one processor 406 coupled to the ultrasonic transducer array 404 via a transmit circuit 408 and a receive circuit 410. The processor 404 can be coupled to a memory 412 storing executable instructions for performing the methods described herein. The processor 404 can also be coupled to a user interface 414. The user interface 414 can include a display device (e.g., a monitor, screen, etc.) for displaying measurement data to a user and/or an input device (e.g., keyboard, mouse, joystick, touchscreen, etc.) for receiving user input. The processor 406 can receive and process the user input, e.g., to control the operation of the ultrasonic transducer array 404.

During the transmit phase, the processor 406 can control the transmit circuit 408 to drive the ultrasonic transducer array 404 in order to produce acoustic energy. In some embodiments, the ultrasonic transducer array 404 is a programmable phased array, and the processor 406 is configured to control the delays and/or amplitudes applied to the individual elements of the phased array in order to control the shape, steering, and/or focusing geometry of the generated acoustic energy. For example, the processor can apply a set of delays and/or amplitudes to the transducer array 404 in order to produce toric focusing of push acoustic energy. The toric focusing can generate a push focal region in the material 402 optimized for efficient acoustic radiation force delivery (e.g., a planar focal region having an elevational width greater than an azimuthal width).

The transducer array 404 can also be used to generate and transmit imaging acoustic energy for measuring the response of the material 402 to the push acoustic energy. The focusing geometry of the imaging acoustic energy can be different than the focusing geometry of the push acoustic energy. For instance, during the transmit phase of the imaging procedure, the processor can apply a set of delays and/or amplitudes to the transducer array 404 in order to produce spherical focusing of imaging acoustic energy. The spherical focusing can be generate an imaging focal region in the material 402 optimized for sensitive detection of displacement and/or deformation (e.g., an ellipsoidal focal region). During the receive phase of the imaging procedure, acoustic energy reflected from the material 402 (e.g., in response to transmitted imaging acoustic energy) can be received by the ultrasonic transducer array 404 and converted into electrical signals. In some embodiments, during the receive phase, the processor applies a set of delays and/or amplitude weights to the transducer array 404 in order to produce dynamic focusing of the ultrasonic transducer array 404. During dynamic focusing, the position and/or geometry of the imaging focal region can be dynamically varied in order to focus the transducer 404 at multiple locations in the material 402. The processor can process the signals received from the ultrasonic transducer array 404 via the receive circuit 410 in order to produce measurement data (e.g., an image of the material 402).

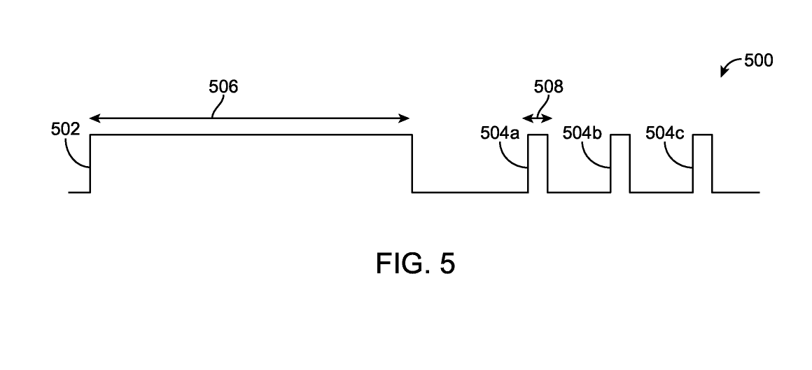

FIG. 5 illustrates a timing diagram 500 for controlling transmission of acoustic energy for producing and measuring displacement of a material, in accordance with embodiments. The timing diagram 500 can be representative of control signals transmitted to an ultrasonic transducer array during a transmit phase in order to generate push acoustic energy and imaging acoustic energy. The control signal can include a push pulse 502 and a plurality of imaging pulses 504a-c. Although FIG. 5 depicts a single push pulse 502 and three imaging pulses 504a-c, it shall be understood that the number of push pulses and imaging pulses can be varied as desired. In some embodiments, the push pulse 502 is delivered first in order to cause the transducer array to generate push acoustic energy for generating material displacement and/or deformation. The duration 506 of the push pulse 502 can be within a range from about 100 μs to about 200 μs. Following the push pulse 502, the imaging pulses 504a-c can be delivered. Each imaging pulse 504a-c can correspond to imaging acoustic energy delivered to a different location in the material so as to image that particular location. The first imaging pulse 504a-c may be delivered no more than about 0.1 μs, 0.5 μs, 1 μs, 5 μs, 10 μs, or 50 μs after the push pulse 502 has terminated. The duration 508 of each imaging pulse can be within a range from about 0.1 μs to about 5 μs.

In some embodiments, the duration 506 of the push pulse 502 is greater than the duration 508 of each imaging pulse, e.g., at least 10 times, at least 20 times, at least 50 times, at least 100 times, at least 500 times, or at least 1000 times greater. The duration of each pulse can be related to the amount of energy delivered to the material by the pulse, such that the push pulse 502 delivers more energy than the imaging pulses 504a-c. Accordingly, the duration 506 of the push pulse 502 can be determined such that the resultant push acoustic energy produces displacement and/or deformation of the material, while the duration 508 of each imaging pulse can be determined such that the resultant imaging acoustic energy does not produce displacement and/or deformation of the material. The duration 506 of the push pulse 502 can be selected such that that the resultant push acoustic energy does not damage and/or destroy the material.

Optionally, the push pulse 502 and/or imaging pulses 504a-c can be repeated. For example, the push pulse 502 can be repeated at least 2 times prior to delivering any of the imaging pulses 504a-c, e.g., in order to produce repeated pushes at the same location in the material. The repetition rate of the push pulse 502 can be approximately 1 Hz to about 50 Hz. Alternatively or in combination, the imaging pulses 504a-c can each be repeated at least 20 times prior to the next imaging pulse, e.g., in order to perform multiple measurements at the same location. The repetition rate of each imaging pulse can be within a range from approximately 1 kHz to about 20 kHz (e.g., for ultrasound imaging), or within a range from approximately 1 kHz to about 250 KHz (e.g., for optical coherence tomography (OCT) imaging).

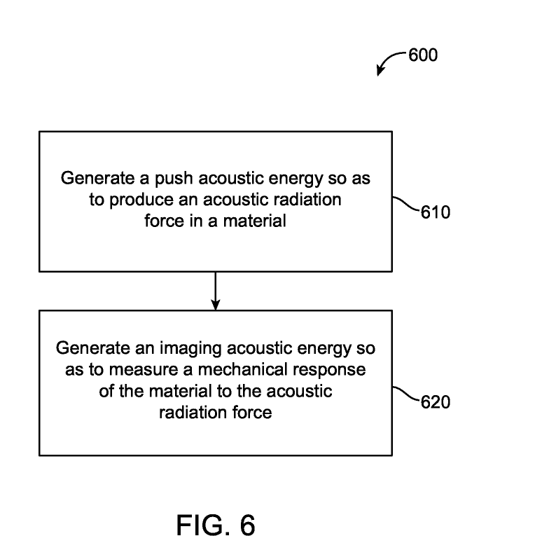

FIG. 6 illustrates a method 600 for producing an acoustic radiation force to generate displacement and/or deformation of a material, in accordance with embodiments. The method 600 can be performed using any embodiment of the systems and devices described herein. In some embodiments, one or more steps of the method 600 are performed using a system for producing and measuring displacement of a material using an ultrasonic transducer array (e.g., the system 400). For example, the system can include one or more processors configured to execute instructions to cause the system to perform the steps of the method 600.

In step 610, a push acoustic energy is generated so as to produce an acoustic radiation force in a material. The acoustic radiation force can generate a mechanical response (e.g., displacement and/or deformation) of the material, such as by producing shear waves). The push acoustic energy can be generated using an ultrasonic transducer array, e.g., an array including a plurality of transducer elements arranged along a first direction and a second direction. The ultrasonic transducer array can focus the push acoustic energy to a push focal region in the material so as to produce the acoustic radiation force. The push focal region can have a geometry optimized for and/or configured to produce the acoustic radiation force, such as an asymmetrical, aspherical, and/or planar geometry. For instance, in some embodiments, the push focal region has a first width along a first direction transverse to (e.g., orthogonal to) a direction of propagation of the push acoustic energy greater than a second width along a second direction transverse to (e.g., orthogonal to) the direction of propagation of the push acoustic energy. The first and second directions can be orthogonal directions. For example, the push focal region can have an elevational width greater than an azimuthal width.

In some embodiments, the ultrasonic transducer array is a programmable phased array, such that the focusing of the push acoustic energy is performed by applying a set of delays and/or amplitudes to the elements of the array. The focusing geometry implemented by the set of delays and/or amplitudes can be optimized for and/or configured to produce the acoustic radiation force to generate the material mechanical response. The set of delays and/or amplitudes can produce toric focusing of the push acoustic energy, for example, in order to generate a planar push focal region with a first focal width different from a second focal width. As described herein, toric focusing can produce acoustic radiation force with greater efficiency.

In step 620, an imaging acoustic energy is generated so as to measure a mechanical response (e.g., displacement and/or deformation) of the material to the acoustic radiation force. The imaging acoustic energy can be generated by the same ultrasonic transducer array of step 610. The ultrasonic transducer array can focus the imaging acoustic energy to an imaging focal region in the material so as to measure the mechanical response. In some embodiments, the imaging focal region has a different geometry than the push focal region. For example, the imaging focal region can have a non-planar geometry. The imaging focal region can be generated by spherical focusing of the ultrasonic transducer array (e.g., during a transmit phase) and/or dynamic focusing of the ultrasonic transducer array (e.g., during a receive phase) in order to produce a real-time image of the material. In embodiments where the ultrasonic transducer array is a programmable phased array, the imaging acoustic energy can be generated by applying a set of delays and/or amplitudes to the phased array different from the set of delays and/or amplitudes applied to the array to generate the push acoustic energy.

The timing of steps 610 and 620 can be varied as desired. In some embodiments, the steps 610 and 620 can be temporally coordinated. For example, step 620 can be performed immediately or within a relatively short time interval after step 610. As discussed herein, the use of programmable phased arrays allows the transducer to be dynamically reconfigured using electronic delays and/or amplitudes so as to allow for near simultaneous acoustic radiation force delivery and displacement detection with optimized focusing geometries. In some embodiments, step 620 is performed no more than about 0.1 μs, 0.5 μs, 1 μs, 5 μs, 10 μs, or 50 μs after step 610. The steps of the method 600 can be repeated as desired (e.g., at predetermined time intervals) in order to obtain a plurality of elastography measurements, e.g., at different locations in the material. For example, step 620 can be repeated with the imaging focal region moved to a plurality of different positions in the material in order to image multiple locations. Alternatively or in combination, step 610 can be repeated with the push focal region moved to a plurality of different positions in the material in order to generate mechanical responses at multiple locations.

In some embodiments, rather than using a single ultrasonic transducer array to produce and measure material displacement and/or deformation, the systems, methods, and devices herein can utilize a separate imaging device in order to perform the imaging. The imaging device can be any device suitable for measuring displacement and/or deformation of the material, such as coherent optical systems including an OCT imaging device, a magnetic resonance imaging (MRI) device, an optical interferometry imaging device, or a second ultrasonic transducer (e.g., a single element transducer or a transducer array). The imaging device can selected for the particular elastography application, e.g., the type of tissue being measured. For example, in some embodiments, OCT imaging is advantageous for certain applications, e.g., for measuring eye tissue or skin tissue, endoscopy, etc. The embodiments disclosed herein are well suited for combination with prior methods and apparatus, such as prior OCT imaging apparatus, and can be used to measure transient deformation of one or more tissues of any eye, such as one or more of a cornea, a lens, a lens capsule, a lens cortex, a lens nucleus, or drainage tissue of any eye such as a trabecular meshwork or Schlemm's canal, for example. An OCT imaging device can be a phase-sensitive OCT imaging device, such as a phase-sensitive 48 OCT imaging device utilizing line scanning (e.g., at a 250 kHz line rate), for example.

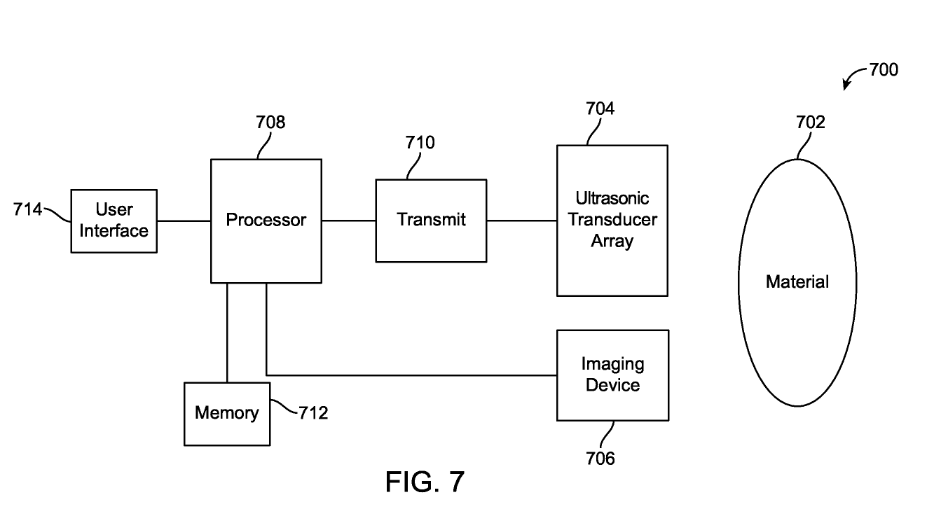

FIG. 7 illustrates a system 700 for producing and measuring displacement of a material 702 using an ultrasonic transducer array 704 and a separate imaging device 706, in accordance with embodiments. The system 700 can also include at least one processor 708 coupled to the ultrasonic transducer array 704 (e.g., via a transmit circuit 710) and to the imaging device 706. The processor 708 can also be coupled to a memory 712 and a user interface 714. The ultrasonic transducer array 704, processor 708, transmit circuit 710, memory 712, and user interface 714 can be similar to the corresponding components described herein with respect to the system 400.

The ultrasonic transducer array 704 can be configured to generate and transmit push acoustic energy to the material 702. As discussed herein, the transducer array 704 can be a programmable phased array, and the processor 708 can be configured to control the delays and/or amplitudes applied to the individual elements of the phased array in order to control the shape, steering, and/or focusing geometry of the generated push acoustic energy. For example, the processor can apply a set of delays and/or amplitudes to the transducer array 704 in order to produce toric focusing of push acoustic energy.

The imaging device 706 can be any imaging device suitable for detecting and measuring the displacement and/or deformation of the material 702 in response to the acoustic radiation force produced by the push acoustic energy. For example, the imaging device 706 can be an OCT imaging device, a MRI device, or a second ultrasonic transducer. The imaging device 706 can be aligned with (e.g., be confocal with) the ultrasonic transducer array 704 in order to measure the displacement and/or deformation generated by the push acoustic energy. The processor 708 can control the ultrasonic transducer array 704 and the imaging device 706 such that the generation of the push acoustic energy is temporally coordinated with the measurements performed using the imaging device 706.

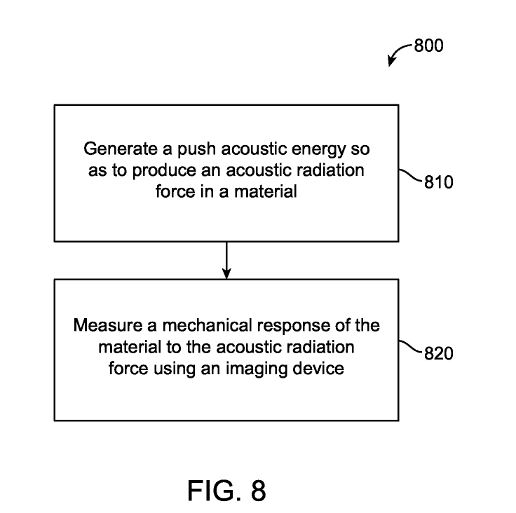

FIG. 8 illustrates a method 800 for producing an acoustic radiation force to generate displacement and/or deformation of a material, in accordance with embodiments. The method 800 can be performed using any embodiment of the systems and devices described herein. In some embodiments, one or more steps of the method 800 are performed using a system for producing and measuring displacement of a material using an ultrasonic transducer array and a separate imaging device (e.g., the system 700). For example, the system can include one or more processors configured to execute instructions to cause the system to perform the steps of the method 800.

In step 810, push acoustic energy is generated so as to produce an acoustic radiation force in a material. Step 810 can be performed similarly to step 610 of the method 600 described herein.

In step 820, a mechanical response (e.g., displacement and/or deformation) of the material to the acoustic radiation force is measured using an imaging device. The imaging device can be separate from the ultrasonic transducer array of step 810. The imaging device can utilize a different imaging modality, such as OCT or MRI. Alternatively, the imaging device can be a second ultrasonic imaging device. The imaging device can be aligned with the ultrasonic transducer array of step 810 in order permit imaging of the mechanical response of the material to the acoustic radiation force.

The timing of steps 810 and 820 can be varied as desired. For example, step 820 can be performed immediately after step 810. The steps of the method 800 can be repeated as desired (e.g., at predetermined time intervals) in order to obtain a plurality of elastography measurements, e.g., at different locations in the material. For example, step 810 can be repeated with the push focal region moved to a plurality of different positions in the material in order to produce a push at multiple locations, while the imaging device remains focused at a single position. This approach may be advantageous in situations where image data can only be obtained from certain locations in the material (e.g., when imaging the lens capsule of the eye).

The following example is included to further describe some aspects of the present disclosure, and should not be used to limit the scope of the invention.

### Example 1

### Toric Focusing for Radiation Force Applications Using a Toric Lens Coupled to a Spherically Focused Transducer

Dynamic elastography using radiation force involves focusing an ultrasound field during hundreds of microseconds at a pressure of several megapascals. This example addresses the importance of the focal geometry. While there may not usually be control of the elevational focal width in generating a tissue mechanical response, a tunable approach can be proposed to adapt the focus geometry that can significantly improve radiation force efficiency. A number of thin, in-house PDMS lenses were designed to modify the focal spot of a spherical transducer. They exhibited low absorption and the focal spot widths were extended up to 8-fold in the elevation direction. Radiation force experiments demonstrated an 8-fold increase in tissue displacements using the same pressure level in a tissue-mimicking phantom with a similar shear wave spectrum, meaning it does not affect elastography resolution. These results demonstrate that larger tissue responses can be obtained for a given pressure level, or that similar response can be reached at a much lower mechanical index (MI). This example provides a proof of concept demonstration applicable to 3 D elastography using 2 D phased arrays where such shaping can be achieved electronically with the potential for adaptive optimization.

The importance of the elevation focus in radiation force applications has often been neglected. Imaging arrays may be relatively efficient and spherical focusing has been used without considering anisotropic focal spots. In this example, an acoustic lens is placed on a spherical single element transducer to change the acoustic focal geometry in one direction only. As lens shape can be calculated from curvatures, the designed lens has two main different curvatures in azimuth and elevation and is equivalent to a section of a torus. Here, a toric lens was designed using simulations of acoustic fields and was manufactured in our lab. Its performance was evaluated in tissue-mimicking phantoms through measurements of displacements near the source and by shear wave speed reconstructions. As discussed in more detail herein, this approach can be used in many fields within the ultrasound community to improve current setups for applications such as ultrasound elastography, MR-ARFI, OCT-elastography, etc. Moreover, the same approach can be done using electronic focusing with 2 D array imaging or therapeutic systems for optimum radiation force emissions.

Materials and Methods

The Toric Lens Coupled to a Focused Transducer

A focused spherical transducer operating at 7.5 MHz central frequency with a focal depth of 35 mm and a diameter of 27.5 mm was used (Sonic Concepts, Seattle, Wash., USA). It can be equivalent to a plane transducer coupled to a lens focusing at a distance f equal to the radius R₀ of the spherical transducer. Let (xz) be the plane of interest for observing the propagation of the shear wave and (yz) be the elevational plane (see FIG. 1). The goal was to modify the focal distance in the (yz) plane while maintaining the same focal distance in the (xz) plane by adding lens focusing at a distance f_{L} in (yz). This involves two curvatures, R_{X} and R_{Y}. Using the lensmaker's equation, the following system of equations can be written:

$$\{ \begin{matrix}f = {R}_{0} & initial transducer \\ 1 \text{/} {f}_{L} = ( n - 1 ) . ( \frac{1}{{R}_{0}} - \frac{1}{{R}_{Y}} + \frac{( n - 1 ) . d}{n . {R}_{0} {R}_{Y}} ) & in ( yz ) \\ 0 = ( n - 1 ) . ( \frac{1}{{R}_{0}} - \frac{1}{{R}_{X}} + \frac{( n - 1 ) . d}{n . {R}_{0} . {R}_{X}} ) & in ( xz )\end{matrix} \qquad ( 1 )$$

where d is the thickness of the lens in the z-axis and n is the refractive index of the lens. The vergence of the lens in (xz) is zero to change the diffracted field in this plane. Combining the transducer and the lens results in equivalent focal depths according to the law of vergence:

$$\{ \begin{matrix}f = {R}_{0} & \begin{matrix}initial \\ transducer\end{matrix} \\ 1 \text{/} {f}_{YZ} = 1 \text{/} {f}_{L} + 1 \text{/} f = \frac{n}{{R}_{0}} - ( n - 1 ) . ( \frac{1}{{R}_{Y}} - \frac{( n - 1 ) . d}{n . {R}_{0} . {R}_{Y}} ) & in ( yz ) \\ 1 \text{/} {f}_{XZ} = 1 \text{/} f = \frac{1}{{R}_{0}} + ( n - 1 ) . ( \frac{1}{{R}_{0}} - \frac{1}{{R}_{X}} + \frac{( n - 1 ) . d}{n . {R}_{0} . {R}_{X}} ) & in ( xz )\end{matrix} \qquad ( 2 )$$

Deriving R_{Y} and R_{X} yields:

$$\{ \begin{matrix}f = {R}_{0} & initial transducer \\ {R}_{Y} = ( n - 1 ) \frac{{f}_{YZ}}{n .} \frac{( n - 1 ) . d - n . {R}_{0}}{{R}_{0} - n . {f}_{YZ}} & in ( yz ) \\ {R}_{X} = {R}_{0} - \frac{( n - 1 ) . d}{n} & in ( xz )\end{matrix} \qquad ( 3 )$$

The focal distance ratio FDR can be defined as the ratio between the focal distances in the (yz) and (xz) planes. A torus can be defined with two radii using a parametric equation:

$$\{ \begin{matrix}{z}_{tor} = - ( R + r . cos ( ϕ ) ) . cos ( θ ) + {R}_{0} \\ {x}_{tor} = ( R + r . cos ( ϕ ) ) . sin ( θ ) \\ {y}_{tor} = ( r . sin ( ϕ ) )\end{matrix} \qquad ( 4 )$$

Setting R+r=R_{X} and r=R_{Y} makes the exterior surface of the torus correspond to the lens surface for θε[−θₘₐₓ;θₘₐₓ] and φε[−φₘₐₓ;φₘₐₓ] where θₘₐₓ and φₘₐₓ are defined by the transducer width.

Manufacturing of Lenses

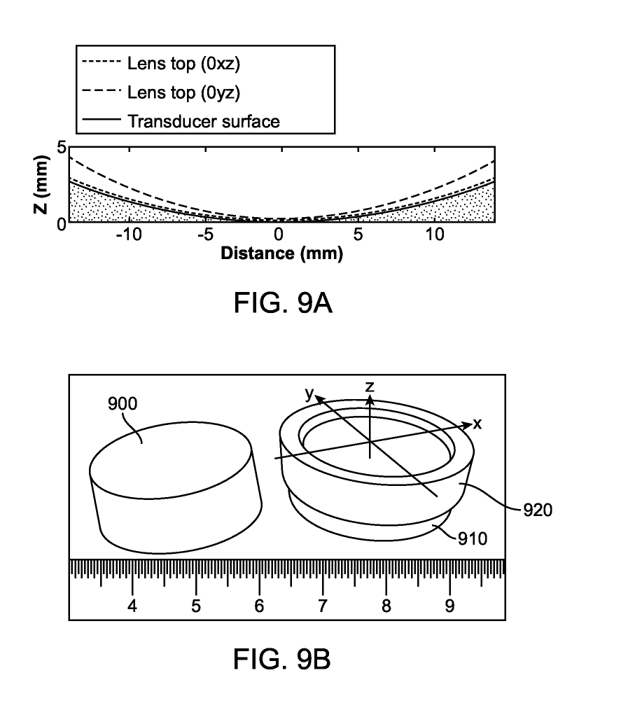

FIGS. 9A and 9B illustrates toric lens geometry for a focal distance ratio of 1.2, in accordance with embodiments. The focus is moved further only in the 0yz direction. FIG. 9A illustrates the lens profile in 2 planes (0xz and 0yz). FIG. 9B illustrates a copy 900 of the transducer shape (left) and a copy 910 with lens 920 mounted on it (right). In this example, different focal distance ratios (FDR) were used: 1.05, 1.1, 1.2 and 1.3. Lens surfaces were computed and corresponding curvatures are shown in FIG. 9A for a FDR of 1.2. The minimal lens thickness was set to 0.2 mm, and the material used was polydimethylsiloxane (PDMS) RTV-615 (MG Chemicals, USA) with a density of 1.02 g/cm³ and a measured speed of sound of 1.020 mm/us. All lenses were made using an aluminum mold machined with a CNC mill, where the molds include a bottom part replicating the transducer shape (see FIG. 9B, left) and a top part corresponding to the lens surface. The two components of PDMS were thoroughly mixed in a 1:10 ratio, and the mixture was then degassed for 1 hour and poured into the bottom part of the mold. The upper part was then pressed on to evacuate surplus PDMS. The mold was placed in an oven at 60° C. for 3.5 hours.

The lens can be designed to be placed directly on the transducer surface, like a cap (see FIG. 9B). It includes a peripheral ring that maintains tight attachment to the transducer. While positioning the lens, bubbles were evacuated by gently pressing the lens against the transducer surface.

Acoustic Simulations

To validate the effect of a toric lens, a semi-analytic simulation of acoustic propagation was programmed using Field II. The surface of the transducer is not considered in the simulation but only the surface of the lens. A definition of a concave transducer is modified to reproduce the top lens surface. Then, the surface was discretized and the delays were set at each surface element (0.5×0.5 mm) to account for propagation delays due to the different lens thicknesses across the aperture. For a FDR of 1.2, the maximum lens thickness was 1.6 mm and the thickness in the center was 0.2 mm. The attenuation through the lens was taken into account with a value of −2.41 dB/mm. The field was calculated in the region of the focus. The simulated field was compared to that measured using a needle hydrophone (ONDA HNC 1000, Sunnyvale, Calif., USA) and 3-dimensional motorized stages.

Tissue Response Model

The dynamic viscoelastic response of tissue subject to radiation force was modeled using the Green's function formalism in the time domain. The analytic expressions of the elastic impulse response to radiation force in a viscoelastic case can be determined using methods known to one of skill in the art. In this approach, the Green's function is the spatio-temporal response to an impulse force in both time and space. As this example concerns the effect of the geometry of the source, the results can be analyzed using convolution by different radiation force spatial patterns \vec{f}(\vec{r}):

$$\vec{u} ( \vec{r} , t ) = {\int }_{τ} d τ {V}_{} \vec{f} ( \vec{ξ} ) . Π ( \frac{τ}{T} ) . \vec{g} ( \vec{r} - \vec{ξ} , t - τ ) . d \vec{ξ} \qquad ( 5 )$$

where \vec{u} is the displacement vector field of the radiation force induced displacements, Π(t/T) is a rectangular function equal to 1 during the push time T, when 0<t<T, and equal to 0 otherwise. \vec{g}(\vec{r}, t) is the sum of 3 Green's function solutions, respectively corresponding to a bulk, a shear and a coupling term. As compressional waves are typically 2 to 3 orders of magnitude faster than shear waves, the displacements observed a few hundreds of microseconds after the push are mainly due to the shear term.

The coupling term can have an effect on the low frequencies observed at the beginning of the wavefront and will be neglected in this example. The shear term is scaled in time with the shear viscosity and in space with the shear speed, or wavelength. As the shear wavelength in soft tissue is typically between 2 and 10 mm depending on the experimental conditions and the stiffness of the medium, there is a regime where the acoustic focal spot can be at least 1 order of magnitude (e.g 0.3 mm) smaller than the shear wavelength. In this regime, the Green's function can be approximated as uniform inside a small Gaussian force source. As the axial focal width (here, 4.6 mm) is greater than the shear half-wavelength, the Green's function will not be considered as uniform in the axial direction.

Consider that the integration of a three-dimensional Gaussian function defined by its variances σ_{x,y,z}² is:

$$I = {V}_{} exp ( - \frac{1}{2} ( \frac{{x}^{2}}{{σ}_{x}^{2}} + \frac{{y}^{2}}{{σ}_{y}^{2}} + \frac{{z}^{2}}{{σ}_{z}^{2}} ) ) . d x . d y . d z = ( 2 π ) . {σ}_{x} . {σ}_{y} . {\int }_{z} exp ( - \frac{1}{2} \frac{{z}^{2}}{{σ}_{z}^{2}} ) . d z \qquad ( 6 )$$

This approximation gives proportionality between the shear displacement and the focal surface in the (XY) plane, when the radiation force is maintained by an equal pressure:

$$\vec{u} ( \vec{r} , t ) \approx {( 2 π )}^{\frac{3}{2}} . {σ}_{x} . {σ}_{y} . {\int }_{τ} d τ \vec{{f}_{0}} . exp ( - \frac{{z}^{2}}{2. {σ}_{z}^{2}} ) .. Π ( \frac{τ}{T} ) . \vec{{g}_{0}} ( \vec{r} - ( \begin{matrix}0 \\ 0 \\ {z}^{′}\end{matrix} ) , t - τ ) . d {z}^{′} \\ \vec{u} ( \vec{r} , t ) ∝ {w}_{x} . {w}_{y} \qquad ( 7 )$$

where w_{x,y,z} is the −3 dB focal width of the acoustic field corresponding to −6 dB values for the force. This approximation may not be valid when the elevation focal size is close to or larger than the shear wavelength. In this regime, a numerical calculation can be used. For each studied lens, numerical calculations have been performed in a 5% gelatin phantom assuming a shear wave speed of 2 m/s, a shear viscosity of 0.2 Pa·s, a push duration of 200 us and imposing a sampling frequency of 12 kHz. The source was sampled in steps of 0.1 mm in the x and y directions and by a step of 0.5 mm in the axial direction based on field 2 simulation results. The field was calculated in the (xz) plane with lateral steps of 1 mm and axial steps of 5 mm.

Shear Wave Elastography Experiments

Shear wave elastography experiments were conducted to evaluate lens performance. The single-element transducer applied radiation force within tissue-mimicking phantoms. A linear array (AT8L12-5 50 mm, elevation focus 20 mm, Broadsound, Taiwan) was connected to a programmable scanner (Verasonics V1, Redmond, Wash., USA) and used to track shear wave propagation.

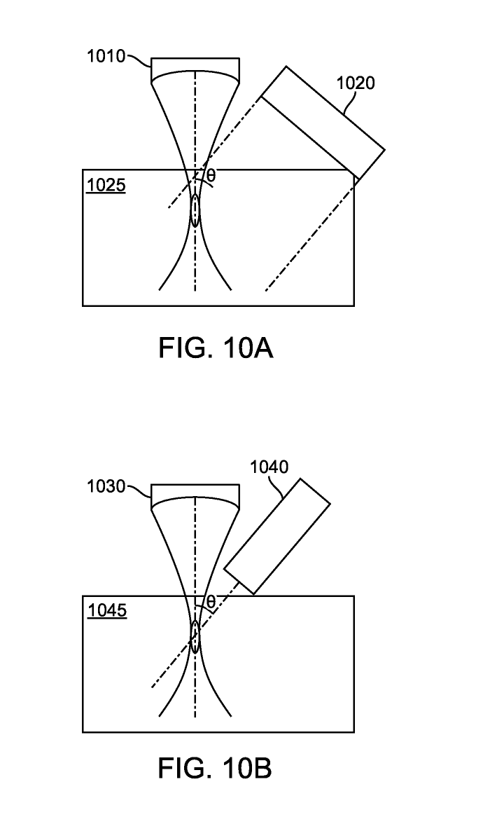

FIG. 10A illustrates configuration and alignment of a single transducer element 1010 and imaging array 1020 for quantification of shear wave amplitudes, in accordance with embodiments. The transducer element 1010 can be a 7.5 MHz focused transducer. The imaging array 1020 can be a L5-12 linear array coupled to a programmable scanner (e.g., a Verasonics scanner, not shown). The imaging plane of the array 1020 can be positioned at an angle θ (e.g., approximately 50°) relative to the axis of the transducer 1010. The quantification can be performed using a 5% gelatin phantom 1025. To study variations in shear wave amplitude, the imaging plane corresponds to the (xz) plane of the focused transducer 1010 as shown in the configuration of FIG. 10A. This configuration enabled imaging of a projection of the shear wave at 50°, meaning that the axial displacements along Z were underestimated by a factor 0.64. If the imaging plane does not correspond to the (xz) plane, the focal spot section would have significantly increased because of the widening in the elevational direction leading to an unfair comparison. In this configuration, the section of the focal spot within the imaging plane should be similar in each case.

FIG. 10B illustrates configuration and alignment of a single transducer element 1030 and imaging array 1040 for shear wave elastography experiments, in accordance with embodiments. The transducer element 1030 can be a 7.5 MHz focused transducer. The imaging array 1040 can be a L5-12 linear array coupled to a programmable scanner (e.g., a Verasonics scanner, not shown). The imaging plane of the array 1040 can be positioned at an angle θ (e.g., approximately 45°) relative to the axis of the transducer 1030. The experiments can be performed using a PVA phantom 1045. To perform an elastography experiment with shear wave speed reconstruction, the transducer 1030 and the array 1040 were placed on the same side of the sample, with an angle of 45° between the transducer axis and the imaging plane, as shown in FIG. 10B. For this configuration, the projection of the shear wave displacements from the axis of the single element to the axial direction of the array (angle of 45°) led to an underestimation of \sqrt{2}/2.

For both configurations, precise alignment was performed using a needle hydrophone (ONDA HNC 1000, Sunnyvale, Calif., USA). It was first positioned at the focus of the single-element transducer. Then, precise positioning of the imaging array was done by maximizing plane wave signals received on the hydrophone and by locating the hydrophone on the ultrasound picture. The hydrophone was then removed to place a tissue mimicking phantom (described below). The coupling medium from the transducer to the phantom was water. The phantom was positioned so that the target was at the focal depths of both the transducer and the imaging array. For the configuration of FIG. 10B, a manual translation stage can move the source laterally for potential reconstruction using multiple shear wave sources.

The single-element transducer was driven by an RF-power amplifier (AR amplifier, USA) using a 200-μs burst (“push”) at a given measured voltage. The transducer was previously calibrated using a lipstick hydrophone (Aperture: 200 um, SEA, Soquel, Calif., USA). Image acquisition includes the repetition of 3 plane waves steered at −5°, 0° and +5° and was triggered simultaneous with the push. A total of 120 frames were recorded at 10 kHz and beamformed using the Verasonics internal beamformer. Coherent plane wave compounding was achieved using a moving average with a kernel size of 3 images. Frame-to-frame displacements (FFD) were calculated using the phase of the cross-correlation function between two frames and bandpass-filtered between 100 Hz and 1500 Hz. The shear wave speed was computed using a time-of-flight algorithm based on zero-phase-crossing correlations. Only results of correlations with a normalized coefficient greater than 0.9 were kept. The absolute displacements were calculated by accumulating the FFD over time.

Tissue Mimicking Phantoms

Two phantoms were used for this study. The first homogeneous phantom was a 5% gelatin solution. The gelatin powder (Sigma Aldrich, USA) was added to deionized water at 80° C. while agitating. When the mixture reached 40° C., it was poured into a square box and stored in the refrigerator at 5° C. for 1 hour.

The second phantom was made of a 10%-PVA (Sigma Aldrich, USA) solution that underwent 2 freeze-thaw cycles (each cycle consists of 6 hours in a −20° C. freezer, followed by 12 hours at ambient temperature). A 2%-agarose (Sigma Aldrich), 5-mm diameter, cylindrical stiff inclusion was embedded in the 10%-PVA background.

For all cases, 1% cellulose (Sigmacell Microcrystalline type 50, Sigma Aldrich) was added to the mixture to provide acoustic scattering.

Results

Simulation Results

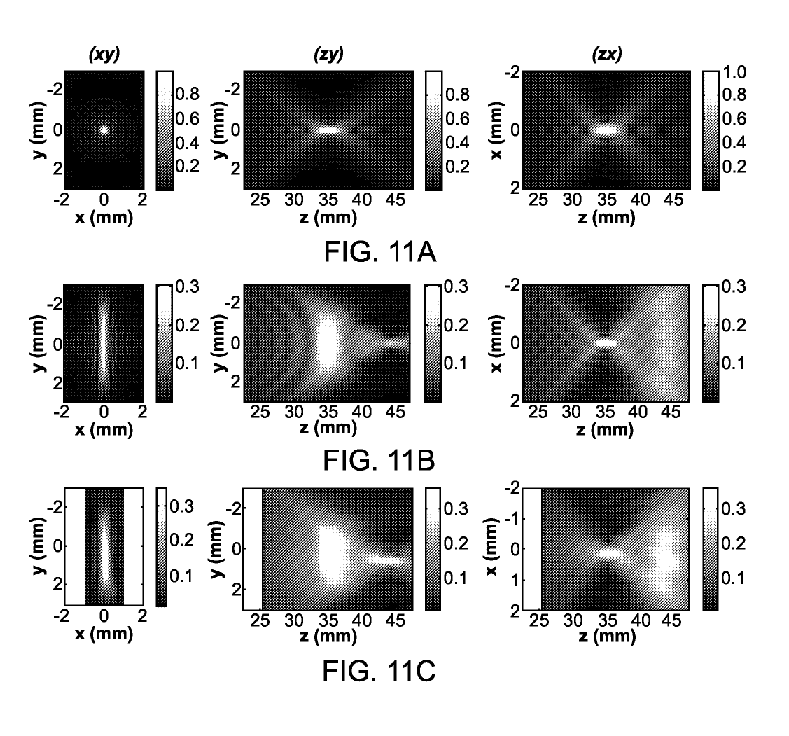

FIGS. 11A through 11C illustrate simulated and experimental acoustic fields, in accordance with embodiments. The pressure is normalized to the case without lens. The acoustic pressure amplitude in the (xy), (yz) and (xz) planes is depicted for the original focus (FIG. 11A), simulated focus with focal distance ratio=1.2 (FIG. 11B), and experimental focus with focal distance ratio=1.3 (FIG. 11C). The original focus of the transducer was first simulated (see FIG. 11A). A −6 dB focal width of 0.35 mm was determined from this simulation. The equivalent patterns for the toric lens (FDR=1.2) are presented in FIG. 11B. The peak amplitude decreased by 11.3 dB due the spread of the acoustic intensity. The attenuation due to the PDMS was found to be negligible. From the secondary focus position in (yz) (see FIG. 11B) the FDR is 1.22 (1.8% error compared to the expected value of 1.2). The simulated focal width in elevation was elongated to 3.4 mm while the azimuth focal width remained unchanged. Note that the side lobes at −13.5 dB in the imaging plane are not important in this case since the radiation force is a function of the pressure squared (i.e., the side lobes produce a radiation force that is reduced by at least 27 dB compared to that from the main lobe).

Fields produced by the toric lenses were scanned experimentally and the elevational focal width for FDR=1.2 was found to be 2.58 mm (24% error compared to the simulation). In the simple model, multiple reflections because of impedance mismatch have been neglected. However, at the PDMS-water interface, 20% of the wave is reflected into the transducer backing. This can change the transducer response across the aperture. Its effect, and the imprecision in manufacturing the lens, could be the reason of the modest deviation between theory and experiment. Nevertheless, an elevational focal width of 3.38 mm was found for the experimental lens with FDR=1.3. In FIG. 11C, the experimental acoustic fields can be compared to the simulated ones in FIG. 11B.

The focus was elongated along the elevational direction only (W_{y}=3.38 mm) while it was not modified along the lateral direction (Wₓ=0.44 mm, same width as the focus without lens). A secondary focus can be identified from the (zy) plane result. The acoustic field in the (zx) plane can also be compared with the one from simulation. The secondary focus is spread in the lateral dimension. Although the experiment gave a higher amplitude for the secondary focus in the (xz) plane, tissue attenuation will be greater than the one from water and will lower the amplitude of the secondary focus of 2.6 dB assuming a typical attenuation of −0.5 dB/cm/MHz (at 7.5 MHz). Then, the radiation force will be lowered by 6.8 dB, yet not totally cancelling the secondary focus potential effects.

The loss in pressure amplitude due to the lens (FDR=1.3) was 9 dB (12% error). These results show that a highly asymmetric focus can be produced with this simple approach.

Shear Wave Elastography Experiments

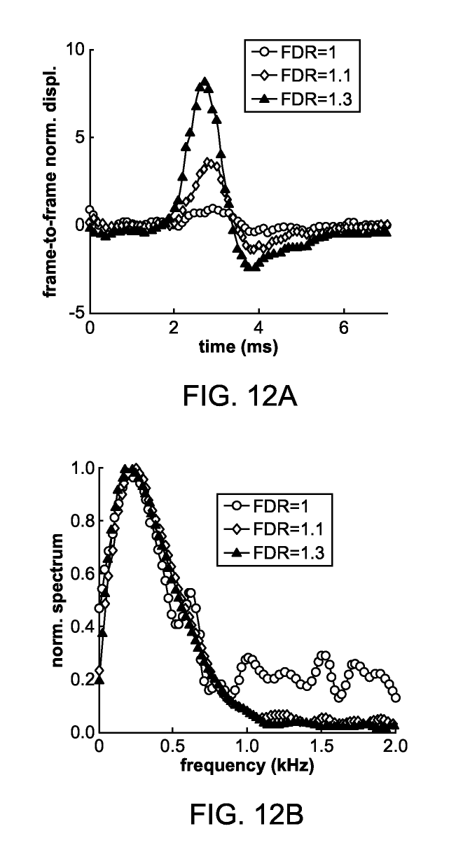

FIGS. 12A and 12B illustrate displacements obtained at a peak pressure of 3.5 MPa in a 5% gelatin phantom using a spherical (circles) and different toric (diamonds, triangles) ultrasound focal spots, in accordance with embodiments. FIG. 12A illustrates frame-to-frame displacements. FIG. 12B illustrates spectral content averaged laterally along a 2 mm line centered at a 4 mm distance from the push and situated at the focal depth of the transducer. Displacements were analyzed 4 mm away from the shear wave source. Without the toric lens (i.e. spherical focus), a peak pressure of 3.5 MPa induced a FFD of 0.057 μm (see FIG. 12A), corresponding to an absolute displacement of 0.436 μm. Different lenses (FDR=1.05, 1.1, 1.2 and 1.3) were then placed on the transducer and the input voltage was respectively increased by a factor of 1.34, 1.74, 2.71 and 2.83 so the maximum pressure matched the initial 3.5 MPa pressure. Using the displacement without lens as the reference, the shear wave peak displacements were respectively multiplied by 0.84, 3.57, 6.49 and 8.1. Since these values are given for positions near the shear source (4 mm away), this improvement is mainly due to the tissue response because effects such as diffraction or absorption are minimized. It is important to note that the spectral contents of the shear waves with/without lens are very similar (see FIG. 12B), meaning that the focal geometry in the (xz) plane mainly governs the spectral content of the shear wave. Furthermore, the presence of a secondary focus did not significantly modify the spectrum of the shear wave. It shows that the tissue response to radiation force can be greatly enhanced (8-fold in this example) at an equivalent MI using a simple approach without requiring a new transducer and without degrading spatial resolution in elastography measurements. Moreover, it demonstrates the benefit of using toric focusing delays for any bi-dimensional phased array system (therapy or imaging).

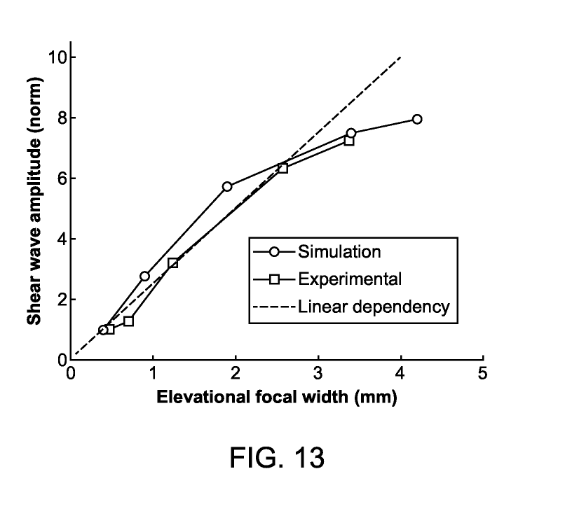

Further, the elastic field generated by a given radiation force pattern was simulated based on a Green's function approach. The 3 D acoustic fields were obtained using Field2 and served as inputs for the radiation force pattern. As this approach provides the tissue response in terms of absolute displacements, the comparison with experimental values is made on cumulative displacements. The normalized displacement amplitude without and with the lens are presented in FIG. 13 as function of the elevational focal width. FIG. 13 illustrates absolute displacement amplitude normalized by the case without lens at a lateral distance of 4 mm from the push as a function of elevational focal width, in accordance with embodiments. Circles indicate Green's function simulation results and diamonds indicate experimental results. The dotted line indicates linear dependency. The experimental and simulation points correspond respectively to FDR=1.0, 1.05, 1.1, 1.2 and 1.3, although with slightly different elevational focal sizes as discussed earlier. Compared in this way, simulation results match closely the experimental ones which show that the elevation focal width dominates the effect of amplification. Further, FIG. 13 demonstrates that the linear dependency supposed in the approximation made previously is valid up to a focal size of 2.5 mm, corresponding to 70% of the shear half-wavelength. Above this value, the Green's function can no longer be considered as uniform throughout the focal spot.

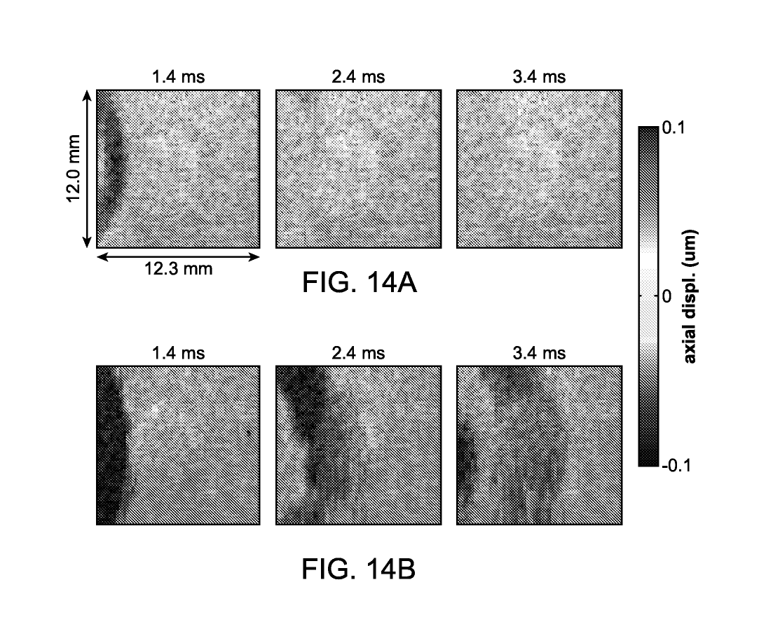

FIGS. 14A and 14B illustrate snapshots of shear wave propagation induced using a spherical (FIG. 14A) and a toric (FIG. 14B) ultrasound focal spot at a peak pressure of 4 MPa, in accordance with embodiments. The sample is a heterogeneous phantom consisting of a 10%-PVA background (2 freeze-thaw cycles) and a 2%-agar inclusion. The shear wave was generated on the left, outside the imaging plane, and propagates to the right. The axial FFD are displayed on a color scale and superimposed on the B-mode image. Snapshots of shear wave frame-to-frame displacement fields are shown without (FIG. 14A) and with the toric lens (FDR=1.2, see FIG. 14B) on the same color scale. For this example, the sample is a heterogeneous phantom containing a stiff inclusion. It appears that the shear wave is detectable over a much larger propagation distance with the lens. For this example, the shear wave propagates with a SNR greater than 10 dB over 5 mm without lens and 11 mm with the lens. In other words, a single shear wave generated with the lens can probe the medium over a larger lateral range than that without the lens. When reconstructing elasticity maps, several shear waves are usually generated at different lateral locations to compensate for this attenuation effect. This result suggests that fewer shear wave source positions are needed to reconstruct the speed at a given lateral range for the toric lens. This enhances the frame rate for real-time display of the elasticity map while maintaining a low I_{SPTA}.

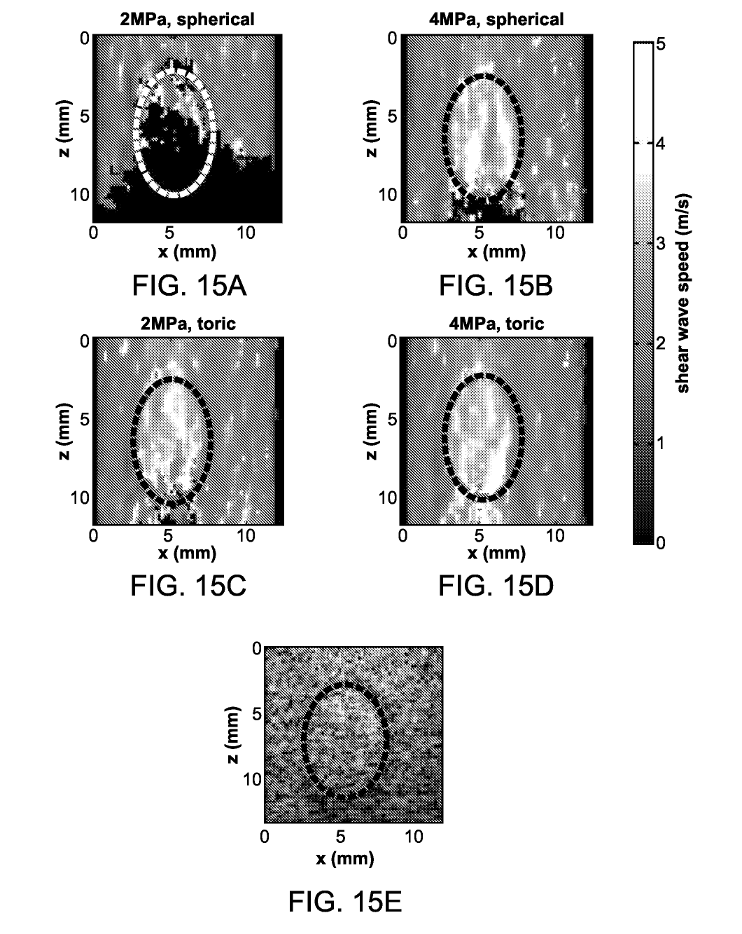

Furthermore, elasticity maps were reconstructed from two shear wave propagation measurements (i.e. two locations of the shear source: respectively left and right of the imaging plane), both repeated without and with the lens (FDR=1.2). FIGS. 15A through 15D illustrate shear wave speed maps of a heterogeneous phantom obtained using a spherical (FIGS. 15A, 15B) and a toric (FIGS. 15C, 15D) ultrasound focal spot at two different peak pressures, in accordance with embodiments. FIG. 15E illustrates a B-mode image of the phantom that consists of a 10%-PVA background (2 freeze-thaw cycles) and a 2%-agar inclusion, in accordance with embodiments. The dashed line delineates the inclusion position determined from the B-mode images.

In FIG. 15E, the inclusion is delineated in the dashed line on the B-mode image. Reconstructions for a pressure of 2 MPa without and with the toric lens are shown in FIG. 15A and FIG. 15C. Using a pressure of 2 MPa (MI=0.73), it was not possible to obtain a full elasticity inversion with two shear waves (see FIG. 15A). The black areas correspond to measurements with a correlation coefficient lower than 0.9. The toric lens enables full elasticity reconstruction at the same pressure (see FIG. 15C). Without the lens, using a pressure of 4 MPa enables almost a full reconstruction similar to the one shown in FIG. 15C. Using both 4 MPa and the lens further improves the quality of the elasticity image (see FIG. 15D).

The region underneath the inclusion exhibited poor speckle amplitude because of a shadowing effect from the inclusion (see FIG. 15E). This degrades the SNR of the detected shear wave in this region and explains artifacts in the elasticity reconstruction for this region.

Discussion

This example presents a new focusing approach that can be easily implemented on existing equipment for any radiation force application. It uses a simple, asymmetric lens to adapt the focal spot geometry of a single-element transducer and improve the radiation force efficiency. A toric lens geometry of a given focal depth aspect ratio was designed and validated using a semi-analytic simulation of acoustic propagation. The resulting field was extended in a single direction that corresponds to the elevation direction for 2 D imaging. Lenses were made of PDMS and mounted on a single element transducer to perform shear wave elastography experiments. The tissue response (e.g., displacement amplitude) obtained was first compared without and with each lens. Using an elevation focus of 3.38 mm, close to the shear wave half-wavelength (3.4 mm), tissue displacements generated in a homogeneous phantom were increased by a factor of 8.1 using a pressure of 3.5 MPa without affecting the shear wave spectrum. Then, the performance of the toric lens in terms of elasticity reconstruction was assessed. It enabled a full elasticity reconstruction using a two-fold smaller acoustic pressure compared to that without the lens and an equal resolution.

The increased displacement amplitude was measured near to the shear source and is due to an enhanced tissue response from the larger acoustic field in the elevation direction. The restoring forces from tissue outside the focus and applied to the displaced area in the focus are moved away to a further extend in the elevation direction. In other words, the shear stresses are reduced in the elevational direction. A larger region is coherently pushed, resulting in a larger tissue response. Radiation force is usually defined as a function of the acoustic intensity via F=α^{P}^{₀}²/2.Z where α is the acoustic absorption, P₀ is the acoustic pressure at the focus and Z is the acoustic impedance of the medium. Here, it can be emphasized that the spatial pattern of the force field is also very important, and most important is the elevational width of the focus. Additional simulations indicated that the decay of the shear wave along its propagation direction in the imaging plane was not affected by the lenses. This means that diffraction is still cylindrical, which is consistent with the elevational width being close to the shear wave half-wavelength.

The resolution for shear wave elastography was not affected by toric focusing in a homogeneous medium. In the presence of a heterogeneity along the extension of the shear source, the spectral content can fluctuate because of a different tissue response along the source. In a dispersive material such as liver, the resulting measured shear wave speed could be slightly changed but this effect is very likely to be minor and not even measurable in practical cases. However, the same situation considered with ARFI-imaging may lead to a change in the resolution of the technique since it is sensitive to the amplitude of the displacements at the focus which may fluctuate along the extension of the shear source. As the resolution is degraded in the elevation direction, the tissue response may be homogenized within this region. This aspect can be considered to determine the tradeoff for ARFI measurements.

For the experiments described here, the shear wave spectrum is conserved because of homogeneity in material properties across the extent of the shear wave source. In other situations, especially involving high frequency shear waves (1000 kHz), toric focusing may have an effect on the spectrum if the source is larger than the half-wavelength. There may be a tradeoff between spatial resolution and amplitude enhancement in highly heterogeneous elastic materials.

This example demonstrates that toric focusing can be applied to a spherical single element transducer using a very thin lens. In this example, a tradeoff has been found between acoustic power and tissue response. An elevational focal width close to the shear half-wavelength produces a significant shear wave amplitude increase. The small gain between FDR=1.2 and 1.3 indicates that the gain saturates and this range of elevational focal size is a good tradeoff between amplification and total acoustic energy.

MI guidelines have been considered as the main limitation on the shear wave source. As acoustic power is increased (up to 8-fold), thermal guidelines also have to be considered in a real-time mode. As the pressure is kept constant, the volume heat source is constant meaning the peak temperature induced by a single push is on the same order. However, thermal diffusion will mainly occur radially in two-dimensions for a spherical focus whereas it will occur mainly along the X-axis for toric focusing resulting in longer relaxation times. Thermal fields simulations using FDR=1.0 and 1.2 have been done using 1-ms pushes at 4 MPa repeated at 2 Hz. The temperature elevation at the focus was almost stationary after 100-s of imaging time and was 4.5 times higher in the toric focusing case compared to the spherical focus. The temperature elevation is thus lower than the increase in acoustic power (11-fold). When required, the pulse repetition frequency (PRF) can be reduced to meet guidelines. However, there are several ways to minimize the potential thermal problem. It is possible to move the shear source to different points in 3 dimensions using a 2 D phased array system. It is also possible to use shorter pushing duration to restore a higher PRF. Eventually, an intermediate FDR can be chosen if it produces a better tradeoff between PRF and amplitude enhancement.

The optimum focal width in elevation to enhance tissue response may depend on the stiffness of the material since the shear wavelength governs diffraction effects. It means that the optimal physical lens or focusing law (for 2 D arrays) may need to be determined for each application (e.g., liver or brain is softer than muscle). Although 3 D printing could be used to build these lenses, resulting surface roughness may be too large compared to the wavelength, at least for high frequency applications. For this example, where the wavelength is close to 200 μm, molds could be made using a CNC mill to decrease surface roughness to less than 20 μm. 3 D printing might offer an alternative, especially for lower frequencies, as long as the resolution and rendering are sufficiently precise (typically ˜λ/10). In any event, 2 D arrays provide the flexibility required to adapt the aspect ratio of the toric lens for a given application, thus optimizing radiation force delivery for a wide range of clinical applications.

A practical approach was proposed to extend the focus in a single direction using toric lenses coupled to the surface of a spherically focused transducer. These lenses were found to be efficient in extending the field to more than 10 times while keeping the other direction intact with a reasonable loss in peak pressure (9 dB). Shear wave elastography experiments were conducted in phantoms and it was found that the lens increased shear wave amplitude by a factor of 8.1. Experimental results were matched to a simulation using elastic Green's functions and matched to a certain extend to a linear approximation developed in theory. It enabled full reconstruction of an elasticity map with only two shear waves using a pressure of 2 MPa (MI of about 0.7). This work can be applied to experimentally optimize shear wave sources for applications such as corneal elastography that require a lower MI (0.23) to satisfy FDA guidelines. Finally, the methods presented herein are well suited to 2 D arrays, where the FDR of the toric lens can be optimized for a given clinical application and the lens can be electronically reconfigured for imaging to sensitively detect resultant displacements.

The various techniques described herein may be partially or fully implemented using code that is storable upon storage media and computer readable media, and executable by one or more processors of a computer system. The processor can comprise array logic such as programmable array logic (hereinafter PAL), configured to perform the techniques described herein. Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

What is claimed is:

1. A system for producing an acoustic radiation force to generate displacement in a material, the system comprising:
an ultrasonic transducer array comprising a plurality of transducer elements arranged along a first direction and a second direction;
one or more processors; and
memory comprising instructions that, when executed by the one or more processors, cause the system to:
generate, using the ultrasonic transducer array, a push acoustic energy focused to a push focal region in the material so as to produce the acoustic radiation force to generate displacement in the material, wherein the push focal region comprises a first width along a first direction transverse to a direction of propagation of the push acoustic energy greater than a second width along a second direction transverse to the direction of propagation of the push acoustic energy.

2. The system of claim 1, wherein the push focal region comprises one or more of an asymmetrical geometry, an aspherical geometry, or a planar geometry.

3. The system of claim 1, wherein the first width comprises an elevational width and the second width comprises an azimuthal width.

4. The system of claim 3, wherein the elevational width is at least 8 times greater than the azimuthal width.

5. The system of claim 1, wherein the push acoustic energy produces a peak acoustic radiation pressure in the material of less than or equal to about 4 MPa.

6. The system of claim 1, wherein the push acoustic energy comprises a frequency of at least 20 MHz.

7. The system of claim 1, wherein the acoustic radiation force produces shear waves that generate the displacement of the material.

8. The system of claim 1, wherein the ultrasonic transducer array comprises one or more of a ring array, an annular array, a 1.25 D array, a 1.5 D array, a 1.75 D array, or a 2 D array of transducer elements.

9. The system of claim 1, wherein the plurality of transducer elements each comprise a characteristic dimension less than an ultrasonic wavelength at a primary operating frequency of the ultrasonic transducer array.

10. The system of claim 1, wherein the ultrasonic transducer array comprises a programmable phased array and the push acoustic energy is generated by applying a set of delays and amplitudes to the programmable phased array configured to produce toric focusing of the push acoustic energy.

11. The system of claim 1, wherein the instructions further cause the system to:
generate, using the ultrasonic transducer array, an imaging acoustic energy focused to an imaging focal region in the material so as to measure the displacement of the material generated by the acoustic radiation force, wherein the imaging focal region comprises a different geometry than the push focal region.

12. The system of claim 11, wherein the push focal region comprises a geometry configured to produce the acoustic radiation force and the imaging focal region comprises a geometry configured to measure the displacement of the material.

13. The system of claim 11, wherein the imaging focal region is generated by spherical focusing of the ultrasonic transducer array during a transmit phase and dynamic focusing of the ultrasonic transducer array during a receive phase to produce a real-time image.

14. The system of claim 11, wherein the generation of the push acoustic energy is temporally coordinated with the generation of the imaging acoustic energy.

15. The system of claim 14, wherein the imaging acoustic energy is generated no more than about 1 μs after generating the push acoustic energy.

16. The system of claim 11, wherein the ultrasonic transducer array comprises a programmable phased array, wherein the push acoustic energy is generated by applying a first set of delays and amplitudes to the programmable phased array in order to focus the push acoustic energy and the imaging acoustic energy is generated by applying a second, different set of delays and amplitudes to the programmable phased array in order to focus the imaging acoustic energy.

17. The system of claim 16, wherein the first set of delays and amplitudes is arranged to produce toric focusing of the push acoustic energy and wherein the second set of delays and amplitudes is arranged to produce spherical focusing of the imaging acoustic energy.

18. The system of claim 1, further comprising an imaging device, wherein the instructions further cause the system to measure the displacement of the material generated by the acoustic radiation force using the imaging device.

19. The system of claim 18, wherein the imaging device is separate from the ultrasonic transducer array.

20. The system of claim 19, wherein the imaging device comprises an optical coherence tomography (OCT) imaging device.

21. The system of claim 19, wherein the imaging device is aligned with the ultrasonic transducer array in order to measure the displacement of the material.

22. The system of claim 18, wherein the generation of the push acoustic energy using the ultrasonic transducer array is temporally coordinated with the measurement of the displacement of the material using the imaging device.

23. The system of claim 18, wherein the instructions further cause the system to move the push focal region to a plurality of different positions in the material.

24. A method for producing an acoustic radiation force to generate displacement in a material, the method comprising:
generating, using an ultrasonic transducer array, a push acoustic energy focused to a push focal region in the material so as to produce the acoustic radiation force to generate displacement in the material, wherein the push focal region comprises a first focal width along a first direction transverse to a direction of propagation of the acoustic energy greater than a second focal width along a second direction transverse to the direction of propagation of the acoustic energy.

25. One or more non-transitory computer-readable storage media having stored thereon executable instructions that, when executed by one or more processors of a system for producing an acoustic radiation force to generate displacement in a material, cause the system to:
generate, using an ultrasonic transducer array, a push acoustic energy focused to a push focal region in the material so as to produce the acoustic radiation force to generate displacement in the material, wherein the push focal region comprises a first focal width along a first direction transverse to a direction of propagation of the acoustic energy greater than a second focal width along a second direction transverse to the direction of propagation of the acoustic energy.
